(19) United States
(12) Patent Application Publication
(10) Pub. No.: US 2023/0263454 A1
(43) Pub. Date: Aug. 24, 2023

(54) ENDOTRACHEAL TUBE FOR INTRA-OPERATIVE NEUROMONITORING

(71) Applicant: inomed Medizintechnik GmbH, Emmendingen (DE)

(72) Inventors: Rudi Mattmüller, Emmendingen (DE); Damir Pfau, Umkirch (DE); Florian Neumeister, Freiburg (DE)

(21) Appl. No.: 18/015,872

(22) PCT Filed: Jun. 16, 2021
(86) PCT No.: PCT/EP2021/066264

§ 371 Date: Jan. 12, 2023

(30) Foreign Application Priority Data
Jul. 15, 2020 (DE) 102020208791.1

(51) Int. Cl. A61B 5/00; A61B 5/395; A61B 5/296; A61B 1/267; A61B 5/257; A61B 5/294; A61B 5/388; A61M 16/04

(52) U.S. Cl. CPC A61B 5/4041; A61B 5/395; A61B 5/296; A61B 1/2673; A61B 5/257; A61B 5/294; A61B 5/388; A61B 5/6847; A61M 16/0434

(57) ABSTRACT

The present invention relates to endotracheal tubes for intra-operative monitoring of nerve and muscle tissue (neuromonitoring), a system for intra-operative neuromonitoring, a method for deriving stimulus responses in intra-operative neuromonitoring, and a method for classifying tissue types, having the purpose of preventing damage to nerves and muscles which run within the area of operation.

### FIELD OF THE INVENTION

The present invention relates to endotracheal tubes for intra-operative monitoring of nerve and muscle tissue (neuromonitoring), a system for intra-operative neuromonitoring, a method for deriving stimulus responses during intra-operative neuromonitoring, and a method for classifying tissue types, in order to prevent damage to nerves and muscles within the area to be operated.

### TECHNICAL BACKGROUND

Intra-operative neuromonitoring is a well-established method for monitoring nerve and muscle responses during surgical procedures in neurosurgery, general surgery and ENT surgery, such as thyroid surgery in the neck. Intra-operative neuromonitoring is used during neck surgery in order to locate and continuously monitor nerve and muscle tissue in the area to be operated to prevent damage thereto during surgery. In surgical procedures on the thyroid gland (goiter), such as partial removal of the thyroid gland (subtotal thyroidectomy) or its complete removal (total thyroidectomy), intra-operative monitoring of nerve and muscle tissue is increasingly used, for example, to enable monitoring of the vocal cord nerve, laryngeus recurrens (NLR).

It is particularly important not to injure the NLR during such interventions. Possible consequences of a unilateral damage to the NLR for the patient include vocal cord disorders including hoarseness and difficulty swallowing, and even permanent vocal cord paralysis (recurrent paresis) with loss of voice. If the NLRs on both sides are injured in the course of thyroid surgery, the paralysed vocal folds (i.e. Musculus vocalis) can close the trachea; this means danger to the patient's life.

Continuous intra-operative monitoring of nerve and muscle tissue during thyroid surgery can identify the course of essential muscles and nerves, such as those of the NLR, in the area to be operated and continuously monitor their function.

In intra-operative neuromonitoring, for example during thyroid surgery, the target nerve, for example the vagus nerve (and optionally the NLR), is stimulated at the beginning of surgery and during manipulation of the target tissue or target organ, for example the thyroid gland. The stimulation of the target nerve is particularly performed electrically.

The vagus nerve is stimulated with a hand-held stimulation probe or with a cuff-shaped electrode wrapped around the vagus nerve. For this purpose, the surgeon places a stimulation probe, for example a rod-shaped stimulation probe, or, for continuous function control, a cuff-shaped electrode around the vagus nerve and stimulates it, for example electrically. In order to identify the course of the nerve, for example the NLR, the surgeon scans the surgical field with a stimulation probe. The step of electrically stimulating of the nerves generates excitation and stimulus transmission to the target tissue or organs, which in a thyroid operation are the vocal folds.

At the same time, an electromyogram (EMG) is recorded from the target area or organ, in this case the vocal folds, and its stimulus response is recorded. The EMG is recorded at the vocal folds by means of an endotracheal tube with surface electrodes on the outside of the tube. The endotracheal tube used for EMG derivation at the vocal folds for oral intubation for the supply of the respiratory system in combination with surface electrodes usually consists of a tube and at least one inflatable element. At least one surface electrode is attached to the outer surface of the tube for conduction, which is in direct contact with the vocal folds. This surface electrode has the advantage that the potentials are conducted directly on the surface of the target muscle.

Alternatively, needle electrodes can be positioned in the patient's vocal folds, in particular by piercing the needle electrodes through the cricoid (cricoid cartilage of the laryngeal skeleton) to record the stimulus response. Based on experience it has shown that the use of needle electrodes for conduction as an alternative to surface electrodes on a tube is associated with bleeding at the puncture site, and it is not seldom that it is associated with incorrect placement and thus incorrect conduction. The exact placement of the electrode on the vocalis muscle depends mainly on the experience of the user. Incorrect placement of a needle electrode can result in damage or perforation of the inflatable element on the tube or the tube itself. Furthermore, the needle electrode can only be placed after the thyroid cartilage has been exposed, which means additional tissue damage for the patient.

The stimulation probe or the cuff-shaped electrode as well as the conduction electrodes (surface electrodes of the endotracheal tube or needle electrodes) are connected to a neuromonitoring device (intra-operative (neuro) monitoring system, IOM system). By means of a nerve monitor, the response signal or EMG is displayed to the surgeon as a stimulus response in real time for examination and interpretation.

Continuous intra-operative neuromonitoring during thyroid surgery can detect nerve damage to the NLR at an early stage, and can prevent bilateral recurrent paresis.

In order to provide the monitoring of nerve and muscle tissue during thyroid surgery as patient-friendly as possible, it is desirable that the attachment of additional probes and electrodes for stimulation and/or conduction becomes unnecessary. To date, there is no method or device which allows both conduction and stimulation with only one instrument or accessory.

### SUMMARY OF THE INVENTION

It is an object of the present invention to improve intra-operative monitoring of potentially vulnerable nerves and muscles, particularly the recurrent laryngeal nerve (NLR), during thyroid surgery, and to make it as gentle on the patient as possible, while allowing precise stimulation of target tissues and derivation of stimulus responses from stimulated target tissues. In addition, in the event of unintentional displacement or rotation of an endotracheal tube used for this purpose, precise stimulation and conduction should still be ensured, so that nerve and muscle tissue can be protected from unintentional damage with a high degree of safety.

Therefore, the present invention provides endotracheal tubes for intra-operative neuromonitoring, a system for intra-operative neuromonitoring, a method for deriving stimulus responses during intra-operative neuromonitoring, and a method for classifying tissue types having the features of the independent claims. Advantageous configurations and further embodiments of the present invention are the object of the respective dependent claims.

Endotracheal Tube with Stimulation Electrodes, Conduction Electrodes and/or Combination Electrodes

According to a first aspect of the present invention, an endotracheal tube for intra-operative neuromonitoring comprises a substantially circular cross-section tube, a fixation element and at least two electrodes. The tube is configured to be inserted into a trachea of a patient. The fixation element is configured to fix the tube in the trachea of the patient when the tube is inserted into the trachea. The at least two electrodes are formed as stimulation electrodes for electrical stimulation of tissue or as conduction electrodes for conduction of stimulus responses from tissue or as combination electrodes for electrical stimulation of tissue and for conduction of stimulus responses from tissue. The at least two electrodes form at least one electrode array. The at least one electrode array is arranged at a predefined array distance from a distal end of the tube at the tube exterior. The at least one electrode array completely surrounds the tube outer surface. The at least two electrodes are arranged in the at least one electrode array such that they are spaced from their respective adjacent electrode at a predefined electrode spacing and substantially parallel to each other, being circular over an area of 360° [degrees] around the tube.

The endotracheal tube according to the first aspect of the present invention is configured for stimulation and for conduction. To this end, at least one of the two electrodes is a stimulation electrode and at least one other of the two electrodes is a conduction electrode. Alternatively, instead of the one stimulation electrode or the one conduction electrode, at least one of the electrodes can be a combination electrode.

The electrodes together form the electrode array. In the electrode array, the electrodes are arranged parallel to each other and at the same electrode spacing. Thus, each of the electrodes has the same electrode spacing to its respective two neighbouring electrodes which are arranged in parallel. Preferably, the stimulation electrodes, the conduction electrodes and the combination electrodes are of the same length. Furthermore, all electrodes preferably are of the same length. It is particularly preferred, that the stimulation electrodes, the conduction electrodes and the combination electrodes have the same geometric shape. In particular, all electrodes preferably have the same geometric shape. Insofar as different types of electrodes (stimulation electrodes and conduction electrodes or stimulation electrodes and combination electrodes or combination electrodes and conduction electrodes or stimulation electrodes and conduction electrodes and combination electrodes) are included, these are preferably arranged alternately in the electrode array. For example, the electrodes can be formed as square platelets of substantially equal-length, being arranged parallel to each other.

The electrodes or the electrode array formed by the electrodes is arranged on the outside of the tube. In this case, the electrode array formed by the electrodes is arranged at the predefined array distance with respect to the distal end of the tube (tube tip). In particular, a distal end of the array is arranged at the array distance to the distal end of the tube or the tip of the tube. The array spacing can be 70 mm [millimetres] to 90 mm. In this case, the electrode array is arranged further proximally than the balloon on the outside of the tube.

The electrodes are arranged in the electrode array in such a way that they i.e. the electrode array completely surrounds the tube outer surface in the circumferential direction. For this purpose, the electrodes in the electrode array are arranged in a 360° circular distribution around the tube. The tube is thus completely surrounded in the area of the electrode array by electrodes spaced apart at the same distance, being arranged parallel to each other. This has the advantage that, despite rotation or displacement of the endotracheal tube, a high signal quality is achieved, and that artefacts are minimised as far as possible.

If at least two stimulation electrodes or at least two combination electrodes or at least one stimulation electrode and at least one combination electrode are present, bipolar stimulation of the (nerve) tissue can take place by means of these electrodes. In this case, a separate counter electrode is not mandatory (but nevertheless possible).

According the endotracheal tube of the present invention, nerves and muscles can be stimulated, and their stimulus responses can be derived at the same time. For this purpose, a combination of an endotracheal tube and surface electrodes is used, which basically comprises a tube, a fixation element such as an inflatable element (low-pressure cuff/balloon) and electrodes on the outside of the tube. For a stable conduction of EMG signals especially at the vocal muscles, the endotracheal tube comprises circularly distributed electrodes for EMG conduction especially at the vocal folds. This type of electrode arrangement ensures continuous and twist-proof signal stability, especially in the event of vertical dislocation (displacement) or rotation of the endotracheal tube.

Due this non-invasive instrument, the surgical field is free of sharp stimulation or conduction instruments that could cause additional damage, especially to the patient's mucous membranes (muscle tissue).

In particular, nerve tissue running through or behind the muscle tissue of a patient can be (electrically) stimulated by means of the electrodes (e.g. fully integrated or applied) arranged on the tube of the endotracheal tube, and a corresponding stimulus response can be derived simultaneously at the vocal folds. In particular, using a suitable intra-operative monitoring system (IOM system), the function of nerves, especially the vagus nerve and the laryngeal recurrent nerve (NLR), can be continuously monitored intra-operatively so that even slight injuries and lesions can be detected and indicated as early as possible, thereby preventing further consequences accordingly.

The lead or combination electrode(s) on the outer surface of the endotracheal tube make it unnecessary to attach another conduction electrode, especially in the form of a needle electrode, at or near the area to be monitored.

The endotracheal tube is pushed into the patient's trachea via the mouth, and the electrodes (stimulation/conduction/combination electrode(s)) of the endotracheal tube are placed in direct contact with the vocal folds. The muscle tissue is then stimulated by means of the stimulation or combination electrode(s) of the endotracheal tube. Additionally or alternatively, the laryngeal adduction reflex (LAR) can be triggered by electrical stimulation of the NLR. The respective response potential, i.e. the stimulus response, is derived from the vocal folds by means of the conduction or combination electrode(s) of the endotracheal tube in the form of an electromyogram. In this way, the vagus nerve or the NLR can be continuously monitored during the operation, and damage to it can be prevented, which can have considerable consequences for the patient.

The endotracheal tube having stimulation electrodes and conduction electrodes, and additionally or alternatively whaving combination electrodes, which are arranged in an electrode array that circularly encloses the tube, enables robust and precise stimulation of target tissue ((nerve) tissue), or conduction of stimulus responses from stimulated target tissue (nerve tissue), since one of the electrodes is always in contact with the target tissue even in the event of (unintentional) rotation or displacement of the endotracheal tube.

Endotracheal Tube with Lining

In accordance with a second aspect of the present invention, an endotracheal tube for intra-operative neuromonitoring comprises a substantially circular cross-section tube, a fixation element, at least one electrode and at least one padding. The tube is configured to be inserted into a trachea of a patient. The fixation element is configured, when the tube is inserted into a trachea of a patient, for fixing the tube in the trachea. The at least one electrode is arranged on an outer surface of the tube. The at least one padding is associated with the at least one electrode and has elasticity in the radial direction. The at least one padding is attached to an underside of the at least one electrode. The at least one padding supports the at least one electrode on the tube exterior in such a way that under the influence of force in the radial direction the at least one padding is elastically deformed, and that the at least one electrode can be displaced in the radial direction and such that, when the force is removed the at least one padding can deform back, the at least one electrode can be displaced back.

The endotracheal tube according to the second aspect of the present invention is configured for stimulation and additionally or alternatively for conduction. For this purpose, at least one electrode is arranged on the tube exterior. The at least one electrode can be a stimulation electrode for electrically stimulating tissue, or a conduction electrode for deriving stimulus responses from tissue, or a combination electrode for electrically stimulating tissue and deriving stimulus responses from tissue.

According to a further embodiment, the endotracheal tube according to the first aspect of the present invention further comprises at least one padding. The padding is associated with at least one of the at least two electrodes, and exhibits elasticity in the radial direction. The at least one padding is attached to an underside of the at least one electrode. The at least one padding supports the at least one electrode on the tube exterior in such a way that, under the influence of force in the radial direction, the at least one padding deforms elastically, and that the at least one electrode can be displaced in the radial direction, and that, when the force is removed, the at least one padding deforms back and the at least one electrode can be displaced back.

The present invention thus further provides an endotracheal tube comprising a tube, a fixation element such as an inflatable element (low pressure cuff/balloon), and at least one padded electrode for stimulation, and additionally or alternatively, for conduction, wherein the at least one padded electrode can be positioned on a target organ, in particular the vocal folds (directly and sufficiently tightly fitting) so that during a surgical intervention (e.g. thyroid operation) an electrical signal can be induced with high accuracy into the target organ, and can be derived therefrom additionally or alternatively.

The padding allows elastic displacement in the radial direction of the at least one electrode with which it is associated. The padding is formed in such a way that the electrode, on the underside of which it is arranged, can be displaced in the radial direction inwards towards the tube by a force, whereby the padding deforms elastically. As soon as the force no longer acts on the electrode, the padding elastically deforms back and thereby displaces the electrode radially outwards back to its original position.

The at least one electrode is placed on an outer side of the padding, or the padding is attached to the underside of the at least one electrode. The padding mechanically connects the at least one electrode to the outside of the tube. For this purpose, the at least one electrode can be applied to the outside of the lining, for example by screen printing, dispensing, piezo jetting or thermobonding. In this way, there are no sharp edges that could injure the patient's mucosa, for example.

The lining can be arranged circularly on the outside of the tube between the tube and the at least one electrode. The padding can be formed separately for each individual electrode or in combination for multiple electrodes. For example, the padding can be formed as a ring around the outside of the tube and support several electrodes against the tube, whereby the electrodes can be attached to the padding with their undersides.

The padding is slightly elastically compressed by the anatomy of the trachea when the endotracheal tube is inserted into the trachea. As a result, the padding exerts a radially outward directed force on the at least one padded electrode, which presses the at least one electrode against the wall of the trachea, so that once the endotracheal tube is correctly positioned in the trachea, the at least one electrode is in optimal contact with the vocal folds.

For the lining, an elastic or flexible material is used that is easily compressible and returns to its original shape on its own. The lining can be compared to ordinary ear plugs. These are compressed before insertion into the ear so that they slide more easily into the ear canal. After the earplugs are positioned, they adapt to the anatomy of the ear canal so that the eardrum is largely sealed from incoming sound and the ear canal is protected from external influences. A similar mechanism is applied to the at least one electrode on the outside of the tube. When the endotracheal tube is inserted, the at least one electrode is pressed against the outside of the tube by the anatomy of the trachea and the tube is inserted into the trachea. After correct placement, the padding will self-expand so that the at least one electrode is in contact with the vocal folds or tissue with optimal pressure. Thus, once the endotracheal tube has been inserted, the at least one electrode is pressed by the padding against the tissue to be stimulated or drained. In the process, the padding gains volume and conforms to the anatomy in such a way that the at least one electrode has optimal contact with the tissue.

When stimulating the (nerve) tissue, this optimal contact, which is achieved by the padding, leads to improved electrical contact and a more stable impedance behaviour. When deriving the stimulus response of the stimulated nerve tissue, the improved contact achieves a higher signal quality. If the position of the endotracheal tube is changed, the geometric shape of the underpadding also changes accordingly, so that the at least one electrode to which the underpadding is assigned is also pressed optimally against the tissue in the new position. Thus, there is always an effective adaptation of the at least one electrode to the tissue that is to be stimulated and additionally or alternatively to the tissue that is to be drained.

System with Endotracheal Tube and Separate Conduction Electrode

According to a third aspect of the present invention, a system for intra-operative neuromonitoring comprises the endotracheal tube according to the first or second aspect of the present invention, and at least one separate conduction electrode. At least one electrode is configured as a stimulating electrode for electrically stimulating tissue or as a combination electrode for electrically stimulating tissue and deriving stimulus responses from the tissue. The at least one separate conduction electrode is configured to be attached on the patient in the vicinity of the stimulated tissue and to derive stimulus responses from the stimulated tissue.

According to a further embodiment of the system, the system comprises at least one counter electrode. The (optional) at least one counter electrode is configured to be attached externally to the patient and to stimulate the tissue monopolar in combination with the at least one stimulation electrode or the at least one combination electrode.

The at least one separate conduction electrode is not included in the endotracheal tube. The electrode is placed on a patient at a different location than the conduction electrode(s) or combination electrode(s) of the endotracheal tube. The separate conduction electrode can be used to record the stimulus response of stimulated nerve tissue on the outside of the endotracheal tube instead of or in addition to the conduction electrode(s) or combination electrode(s) of the endotracheal tube. The conduction of the stimulus response is preferably performed optionally by means of at least one of the conduction electrodes of the endotracheal tube or alternatively by means of the at least one separate conduction electrode.

The at least one separate conduction electrode enables a particularly simple and yet robust conduction of stimulus responses of the stimulated nerve tissue.

The (optional) at least one counter electrode is not included in the endotracheal tube. The electrode is attached to the patient at a different location than the stimulation electrode(s) or combination electrode(s) of the endotracheal tube and serves as a counter pole to the stimulation pole(s) formed by the stimulation electrode(s)/combination electrode(s). In particular, the (nerve) tissue can be stimulated in a monopolar fashion by means of a stimulation electrode or combination electrode of the endotracheal tube on its tube outer side (a stimulation pole) and a counter electrode (counter pole).

Alternatively, the counter electrode is comprised of a stimulation electrode or combination electrode located on the outside of the tube, or is further defined alternatively from an interconnection of several of the electrodes on the outside of the tube. The monopolar stimulation of (nerve) tissue is carried out accordingly by means of that counter electrode on the outside of the tube and a further stimulation electrode or combination electrode.

The (optional) at least one counter electrode enables a particularly simple and yet robust stimulation of (nerve) tissue.

Method for Deriving Stimulus Responses in Intra-Operative Neuromonitoring

According to a fourth aspect of the present invention, a method for deriving stimulus responses in intra-operative neuromonitoring with an endotracheal tube according to the first or second aspect of the present invention or a system according to the third aspect of the present invention comprises the steps of:
Electrically stimulating tissue, in particular muscle tissue, by means of at least one stimulation electrode or at least one combination electrode of the endotracheal tube.
Deriving a stimulation response of the stimulated tissue, in particular the stimulated muscle tissue, preferably at the vocal folds, by means of at least one conduction electrode or at least one combination electrode of the endotracheal tube or by means of at least one separate conduction electrode of the system.

According to a further development of the derivation method, the step of electrically stimulating of tissue is additionally performed by means of at least one counter electrode of the system.

The advantages, as described above, of the endotracheal tubes according to the first and second aspects of the present invention and of the system according to the third aspect of the present invention are also achieved in an analogous manner with the method according to the fourth aspect of the present invention.

Endotracheal Tube with Padding

In accordance with a second aspect of the present invention, an endotracheal tube for intra-operative neuromonitoring comprises a substantially circular cross-section tube, a fixation element, at least one electrode and at least one padding. The tube is configured to be inserted into a trachea of a patient. The fixation element is configured, when the tube is inserted into a trachea of a patient, to fix the tube in the trachea. The at least one electrode is arranged on an outer surface of the tube. The at least one padding is associated with the at least one electrode and has elasticity in the radial direction. The at least one padding is attached to an underside of the at least one electrode. The at least one padding supports the at least one electrode on the tube exterior in such a way that under the influence of force in the radial direction the at least one padding is elastically deformed and the at least one electrode can be displaced in the radial direction, and when the force is removed the at least one padding can deform back and the at least one electrode can be displaced back.

The endotracheal tube according to the second aspect of the present invention is configured for stimulation and additionally or alternatively for conduction. For this purpose, at least one electrode is arranged on the tube exterior. The at least one electrode can be a stimulation electrode for electrically stimulating tissue, or a conduction electrode for deriving stimulus responses from tissue, or a combination electrode for electrically stimulating tissue and deriving stimulus responses from tissue.

According to a further embodiment, the endotracheal tube according to the first aspect of the present invention further comprises at least one padding. The padding is associated with at least one of the at least two electrodes and exhibits elasticity in the radial direction. The at least one padding is attached to an underside of the at least one electrode. The at least one padding supports the at least one electrode on the tube exterior in such a way that, under the influence of force in the radial direction, the at least one padding deforms elastically and the at least one electrode can be displaced in the radial direction and, when the force is removed, the at least one padding deforms back and the at least one electrode can be displaced back.

The present invention thus further provides an endotracheal tube comprising a tube, a fixation element such as an inflatable element (low pressure cuff/balloon) and at least one padded electrode for stimulation. Endotracheal tube comprising a tube pipe, a fixation element such as an inflatable element (low-pressure cuff/balloon) and at least one padded electrode for stimulation and additionally or alternatively for derivation, wherein the at least one padded electrode can be positioned on a target organ, in particular the vocal folds (directly and sufficiently tightly fitting so that during a surgical intervention (e.g. thyroid operation) an electrical signal can be induced with high accuracy into the target organ and additionally or alternatively or derived therefrom.

The lining (padding) allows elastic displacement in the radial direction of the at least one electrode with which it is associated. The padding is formed in such a way that the electrode, on the underside of which it is arranged, can be displaced in the radial direction inwards towards the tube by a force, whereby the padding deforms elastically. As soon as the force no longer acts on the electrode, the padding elastically deforms back and thereby displaces the electrode radially outwards back to its original position.

The at least one electrode lies on an outer side of the padding or the padding is attached to the underside of the at least one electrode. The padding mechanically connects the at least one electrode to the outside of the tube. For this purpose, the at least one electrode can be applied to the outside of the lining, for example by screen printing, dispensing, piezo jetting or thermobonding. In this way, there are no sharp edges that could injure the patient's mucosa, for example.

The lining can be arranged circularly on the outside of the tube between the tube and the at least one electrode. The padding can be formed separately for each individual electrode or in combination for multiple electrodes. For example, the padding can be formed as a ring around the outside of the tube and support several electrodes against the tube, whereby the electrodes can be attached to the padding with their undersides.

The padding is slightly elastically compressed by the anatomy of the trachea when the endotracheal tube is inserted into the trachea. As a result, the padding exerts a radially outward force on the at least one padded electrode, which presses the at least one electrode against the wall of the trachea, so that once the endotracheal tube is correctly positioned in the trachea, the at least one electrode is in optimal contact with the vocal folds.

For the lining, an elastic or flexible material is used that is easily compressible and returns to its original shape on its own. The lining can be compared to ordinary ear plugs. These are compressed before insertion into the ear so that they slide more easily into the ear canal. After the earplugs are positioned, they adapt to the anatomy of the ear canal so that the eardrum is largely sealed from incoming sound and the ear canal is protected from external influences. A similar mechanism is applied to the at least one electrode on the outside of the tube. When the endotracheal tube is inserted, the at least one electrode is pressed against the outside of the tube by the anatomy of the trachea and the tube is inserted into the trachea. After correct placement, the padding will self-expand so that the at least one electrode is in contact with the vocal folds or tissue with optimal pressure. Thus, once the endotracheal tube has been inserted, the at least one electrode is pressed by the padding against the tissue to be stimulated or drained. In the process, the padding gains volume and conforms to the anatomy in such a way that the at least one electrode has optimal contact with the tissue.

When stimulating the (nerve) tissue, this optimal contact, which is achieved by the padding, leads to improved electrical contact and a more stable impedance behaviour. When deriving the stimulus response of the stimulated nerve tissue, the improved contact achieves a higher signal quality. If the position of the endotracheal tube is changed, the geometric shape of the underpadding also changes accordingly, so that the at least one electrode to which the underpadding is assigned is also pressed optimally against the tissue in the new position. Thus, there is always an effective adaptation of the at least one electrode to the tissue that is to be stimulated and additionally or alternatively to the tissue that is to be drained.

System with Endotracheal Tube and Separate Conduction Electrode

According to a third aspect of the present invention, a system for intra-operative neuromonitoring comprises the endotracheal tube according to the first or second aspect of the present invention and at least one separate conduction electrode. At least one electrode is configured as a stimulating electrode for electrically stimulating tissue or as a combination electrode for electrically stimulating tissue and deriving stimulus responses from the tissue. The at least one separate conduction electrode is configured to be attached on the patient in the vicinity of the stimulated tissue and to derive stimulus responses from the stimulated tissue.

According to a further embodiment of the system, the system comprises at least one counter electrode. The (optional) at least one counter electrode is configured to be attached externally to the patient and to stimulate the tissue monopolar in combination with the at least one stimulation electrode or the at least one combination electrode.

The at least one separate conduction electrode is not included in the endotracheal tube. It is placed on a patient at a different location than the conduction electrode(s) or combination electrode(s) of the endotracheal tube. The separate conduction electrode can be used to record the stimulus response of stimulated nerve tissue on the outside of the endotracheal tube instead of or in addition to the conduction electrode(s) or combination electrode(s) of the endotracheal tube. The conduction of the stimulus response is preferably performed optionally by means of at least one of the conduction electrodes of the endotracheal tube or alternatively by means of the at least one separate conduction electrode.

The at least one separate conduction electrode enables a particularly simple and yet robust conduction of stimulus responses of the stimulated nerve tissue.

The (optional) at least one counter electrode is not included in the endotracheal tube. It is attached to the patient at a different location than the stimulation electrode(s) or combination electrode(s) of the endotracheal tube and serves as a counter pole to the stimulation pole(s) formed by the stimulation electrode(s)/combination electrode(s). In particular, the (nerve) tissue can be stimulated in a monopolar fashion by means of a stimulation electrode or combination electrode of the endotracheal tube on its tube outer side (a stimulation pole) and a counter electrode (counter pole).

Alternatively, the counter electrode is comprised of a stimulation electrode or combination electrode located on the outside of the tube or is further defined alternatively from an interconnection of several of the electrodes on the outside of the tube. The monopolar stimulation of (nerve) tissue is carried out accordingly by means of that counter electrode on the outside of the tube and a further stimulation electrode or combination electrode.

The (optional) at least one counter electrode enables a particularly simple and yet robust stimulation of (nerve) tissue.

Method for Deriving Stimulus Responses in Intra-Operative Neuromonitoring

According to a fourth aspect of the present invention, a method for deriving stimulus responses in intra-operative neuromonitoring with an endotracheal tube according to the first or second aspect of the present invention or a system according to the third aspect of the present invention comprises the steps of:
Electrically stimulating tissue, in particular muscle tissue, by means of at least one stimulation electrode or at least one combination electrode of the endotracheal tube.
Deriving a stimulation response of the stimulated tissue, in particular the stimulated muscle tissue, preferably at the vocal folds, by means of at least one conduction electrode or at least one combination electrode of the endotracheal tube or by means of at least one separate conduction electrode of the system.

According to a further development of the derivation method, the step of electrically stimulating of tissue is additionally performed by means of at least one counter electrode of the system.

The described advantages of the endotracheal tubes according to the first and second aspects of the present invention and of the system according to the third aspect of the present invention are also achieved in an analogous manner with the method according to the fourth aspect of the present invention.

Method for Classifying Tissue Types

According to a fifth aspect of the present invention, a method for classifying tissue types comprises the steps of:
Inducing at least one alternating electrical signal with at least two different predefined frequencies (e.g. electrical sinus signal), in particular with at least one further predefined parameter and particularly preferably with predefined amplitude and additionally or alternatively predefined pulse-wise into tissue by means of at least two inducing electrodes, which are in particular formed by stimulation electrodes, conduction electrodes or combination electrodes on a tube outer side of a tube of an endotracheal tube.
Measuring a current waveform and a voltage waveform of the induced alternating electrical signal in the tissue by means of the at least two inducing electrodes or by means of at least two measuring electrodes.
Calculating at least two impedances of the tissue on the basis of the measured current curve and the measured voltage curve.
Classifying a tissue type of the tissue based on the calculated at least two impedances.

According to a further development of the method for classifying tissue types, the at least two inducing electrodes are formed by stimulation electrodes, inducing electrodes or combination electrodes on a tube outer surface of a tube barrel of an endotracheal tube.

Endotracheal Tube for Classifying Tissue Types

According to a sixth aspect of the present invention, an endotracheal tube for classifying tissue types is configured to perform the method according to the fifth aspect of the present invention. The at least two inducing electrodes and optionally the at least two measuring electrodes are arranged on a tube outer surface of a tube barrel of the endotracheal tube.

According to a further development, the endotracheal tube is configured according to the first or second aspect of the present invention to enable the method according to the fifth aspect of the present invention to be performed. At least two of the electrodes are the at least two inducing electrodes. Optionally, at least two further ones of the electrodes are the at least two measuring electrodes.

According to a further aspect, the system according to the third aspect of the present invention is configured to perform the method according to the fifth aspect of the present invention. At least two of the electrodes are either the at least two inducing electrodes or the at least two measuring electrodes. At least two separate dissipation electrodes or counter electrodes are correspondingly either the at least two measuring electrodes or the at least two inducing electrodes.

In order to be able to determine an impedance of the tissue, the tissue type of which is to be categorised, at a certain frequency, an alternating electrical signal (an alternating electrical current) is induced into the tissue at this frequency. A current flows from at least one inducing electrode to at least one other inducing electrode. The inducing electrodes can be stimulation electrodes, inducing electrodes or combination electrodes on the outside of the tube of the endotracheal tube or other electrodes that are in contact with the tissue to be categorised. The alternating electrical signal is preferably a sinusoidal signal, square wave signal, triangular signal or the like.

In order to be able to categorise the tissue type of the tissue, at least two determined impedances at at least two different predefined frequencies of the alternating electrical signal are required. Therefore, either at least two different predefined frequencies are superimposed in the induced alternating signal or the alternating electrical signal is induced with at least two different predefined frequencies in two temporally successive intervals. The alternating electrical signal can be generated by a signal generator or a stimulator. The signal generator can be integrated into an IOM system. In this case, the at least two inducing electrodes can be electrically connected to the signal generator or the IOM system or the stimulator by means of suitable connections.

The voltage resulting from applying the current of the alternating electrical signal, which is present between the inducing electrodes, can be measured by means of a suitable voltage measurement set-up. For this purpose, the at least two single-conductor electrodes themselves or the at least two optional measuring electrodes are used. Either only the voltage or the voltage curve is measured and the current or current curve is calculated from this or, alternatively, the current or current curve is measured simultaneously. The voltage measured with the introduction/measuring electrodes and the measured or calculated current are influenced by the tissue type of the tissue into which the alternating electrical signal was induced. The actual measurement of the voltage and/or current can be performed by a measuring unit. The measuring unit can be integrated into the IOM system. The at least two measuring electrodes can be electrically connected to the measuring unit or the IOM system by means of suitable connections.

The at least two impedances are calculated on the basis of the measured current curve and voltage curve at each of the at least two frequencies of the induced alternating electrical signal. A calculation unit, which can be integrated into the IOM system, can calculate the impedance from the measured voltage waveform and the measured current waveform for each of the frequencies.

Based on the calculated impedances of the tissue, the tissue type of the tissue is then categorised. For example, a look-up table of impedances at different frequencies and corresponding tissue types can be used to categorise the tissue type based on the calculated impedances of the tissue. In particular, target tissue can be categorised as at least either muscle tissue or other tissue based on the calculated impedances. A categorisation unit, which can be integrated into the IOM system, can perform the categorisation of the tissue type based on the calculated impedance of the tissue.

By classifying the tissue type of target tissue, more specifically by differentiating muscle tissue from other tissue, based on the calculated impedances, the location of the endotracheal tube can be identified and optimised by moving the endotracheal tube so that effective stimulation of tissues and low artefact derivation of their stimulus responses can be achieved.

The following description relates to or pertains to all aspects of the present invention.

The endotracheal tube is shaped to be insertable into the trachea of a patient. The cross-section of the tube is substantially round throughout and can have substantially the same diameter throughout. The substantially round cross-section of the tube has an (outer) diameter that is smaller than a diameter of the trachea (varies depending on patient, age, gender). Usually, the inner diameter is given in millimetres. The choice of tube size depends mainly on the patient's age and the width of the subglottic space. Preferably, the inner diameter of the essentially round cross-section is 2 mm-11 mm [millimetres]. The wall thickness of the tube is very small compared to the inner diameter, so that the outer diameter is substantially equal to the inner diameter. The edges of the tube are rounded to prevent damage to the mucous membranes or parts of the larynx during insertion and removal of the endotracheal tube and by turning or changing the position of the tube. The tube is open at both ends. A side opening can also be incorporated into the tube. If the end of the tube is blocked, the patient can be adequately ventilated through the lateral opening of the tube. At the proximal end of the tube, the endotracheal tube can be connected to a ventilator by means of a standardised connector with an optionally integrated valve, and the electrodes can be connected to an IOM system by means of another connector and a connecting cable.

Proximal to the distal end of the tube, the fixation element can be arranged on the outside of the tube at a predefined element distance. After the endotracheal tube has been correctly inserted and placed in the trachea, the tube is fixed in the centre of the trachea by means of the fixation element.

The fixation element can be an inflatable element, retractable hooks and the like. The inflatable element can be, for example, a balloon or a low-pressure cuff. The inflatable element can be at least partially made of silicone and, after placement of the endotracheal tube, can be inflated with low air pressure by means of a thin tube that can first run along the inner wall and further proximally along the outer wall of the tube. The tube can be connected to an air-filled disposable syringe by means of a Luer connection and inflated/inflated and/or deflated through it. By pushing in the plunger of the disposable syringe by a user, the air flows into the inflatable element at low air pressure and inflates it. In doing so, the inflatable element (balloon/cuff) can seal the trachea at the same time.

The risk of unwanted movements of the tube and the associated decrease in signal quality are thus reduced. Furthermore, an inflatable balloon/cuff in particular counteracts aspiration and pressure-related mucosal damage in the trachea area.

Tissue, in particular muscle tissue, can be electrically stimulated by means of the stimulation electrode(s) and additionally or alternatively by means of the combination electrode(s) and optionally additionally the counter electrode(s). For this purpose, corresponding electrical currents (electrical stimulation signals) suitable for stimulating (nerve) tissue can be conducted to the stimulation electrode(s) or combination electrode(s) from an IOM system that can be electrically connected to the electrodes by means of a suitable connection (connector). The stimulation electrode(s) or combination electrode(s) form(s) one/several stimulation pole(s). The counter electrode(s) form(s) one/several counter pole(s) to the stimulation pole(s). By means of the stimulation electrode(s) or combination electrode(s) (stimulation pole(s)), the electrical currents from the IOM system are induced into the tissue opposite one of the further stimulation electrode(s) or combination electrode(s) in a bipolar or multipolar manner or opposite the counter electrode(s) (counter pole(s)) in a monopolar, bipolar or multipolar manner.

One or more stimulation electrodes or combination electrodes in combination can form a stimulation pole. For example, four stimulation electrodes or combination electrodes can in combination form a stimulation pole. Only one, two or three of the four stimulation electrodes or combination electrodes can also form a stimulation pole. Thus, for example, one, two, three or four stimulation poles can be formed with four stimulation electrodes or combination electrodes. Each stimulation pole can introduce a different electrical stimulation signal (each generated by the IOM system) into the (nerve) tissue by means of the corresponding stimulation electrodes or combination electrodes.

The induced electrical currents (electrical stimulation signals) stimulate the muscle tissue in such a way that a response signal, the so-called stimulus response of the tissue, is generated. The stimulus signal is transmitted along the nerve tissue (stimulus conduction) and can be detected along the nerve tissue, even at a site distant from the site of stimulation.

The conduction electrode(s) and additionally or alternatively the combination electrode(s) and optionally also the separate conduction electrode(s) can derive the stimulus response of the stimulated nerve tissue, more precisely the EMG(s). The derived stimulus responses can be transmitted to the IOM system, which can be electrically connected to the electrodes by means of the appropriate connector, for display and additionally or alternatively for further signal processing and additionally or alternatively for signal conditioning.

One or more conduction electrodes or combination electrodes in combination can form a derivative pole. For example, four conduction electrodes or combination electrodes can in combination form a derivative pole. It is also possible for only one, two or three of the four conduction electrodes or combination electrodes to form a conduction pole. Thus, for example, one, two, three or four discharge poles can be formed with four discharge electrodes or combination electrodes. Each lead pole can derive a different lead of a stimulus response (which is transmitted to the respective IOM system) by means of the corresponding conduction electrodes or combination electrodes from stimulated nerve tissue.

The combination electrode(s) is/are used in continuous alternation as stimulation electrode(s) and conduction electrode(s). For this purpose, an electrical stimulation signal from the IOM system is first induced into the (nerve) tissue by means of the combination electrode(s) in a stimulation time interval. Subsequently, a stimulus response of the stimulated (nerve) tissue is derived by means of the combination electrode(s) in a recording time interval. The stimulation interval and the conduction interval follow each other continuously and alternately. The length of the stimulation interval can be equal to or different from the length of the conduction interval. The stimulation interval can preferably be 100 μs [microseconds] to 2000 μs long. The derivative interval can preferably be 100 μs to 500 μs in length.

The step of electrically stimulating signal can be a sinusoidal signal, square wave signal, triangular signal and the like. Preferably, the stimulation signal can have a current of 10 μA [microampere] to 250 mA and a voltage of 1 V [volt] to 410 V) and a pulse width of 50 μs to 30 ms [milliseconds] and a frequency of, 0.1 Hz [hertz] to 1000 Hz.

Preferably, the electrodes can be formed as substantially rectangular strips extending along the outside of the tube. The strips have a length greater than their width and a width greater than their thickness. Preferably, an electrode formed as a strip has a length of 25 mm to 55 mm. Further preferably, an electrode formed as a strip has a width of from 2 mm to 5 mm. Further preferably, an electrode formed as a strip has a thickness of 10 μm to 100 μm.

The endotracheal tube according to the first and second aspects of the present invention and the system according to the third aspect of the present invention are each connectable to an intra-operative monitoring (IOM) system.

The IOM system can comprise a processing unit, a user interface, an output unit, a power supply and a connection interface for connecting the endotracheal tube or the system. The processing unit can comprise one or more data processing devices, such as microcontrollers (μC), integrated circuits, application-specific integrated circuits (ASIC), application-specific standard products (ASSP), digital signal processors (DSP), field programmable gate arrays (FPGA) and the like. In particular, the processing unit can comprise a stimulation unit configured to generate electrical currents for stimulating (nerve) tissue (stimulation signals). Further, the processing unit can comprise a monitoring unit configured to monitor derived stimulus responses. The monitoring unit can be communicatively connected to the stimulation unit. Further, the processing unit can comprise the signal generator. In particular, the stimulation unit can comprise the signal generator. Further, the processing unit can comprise the measurement unit, the determination unit and the calculation unit. In particular, the monitoring unit can comprise the measurement unit, the determination unit and the calculation unit.

The user interface can receive input from a user or user and forward it to the processing unit. In particular, the user interface can comprise a keyboard, a mouse, a joystick, a control panel, a touch screen and the like.

The output unit can output information from the processing unit to the user/user. In particular, the output unit can comprise a screen, a speaker, a touch screen and the like.

The power supply provides electrical power to the processing unit, the user interface, the output unit and endotracheal tubes or systems connected to the connection interface. The power supply can be a power supply unit, a battery or the like.

The connection interface is formed to electrically and additionally or alternatively communicatively connect an endotracheal tube or system to the IOM system. The connection interface can be a socket with multiple connections for pins or the like. The electrical and additionally or alternatively the communicative connection between the endotracheal tube or the system and the IOM system by means of the connection interface can be made by means of a (connection) cable with a corresponding connection (connector), such as a plug with several pins, which can be plugged into the socket of the connection interface. In particular, the stimulation unit can be electrically connected to the stimulation electrode(s) or combination electrode(s) and counter electrode(s) by means of electrical lines in the cable. Furthermore, the monitoring unit can be electrically connected by means of electrical lines in the cable to the dissipation electrode(s) or combination electrode(s) as well as optional separate dissipation electrode(s). Furthermore, the signal generator can be electrically connected to the inducing electrodes and the measuring unit can be electrically connected to the measuring electrodes, each by means of electrical lines in the cable.

According to a further embodiment of the present invention, the at least two electrodes extend axially or circularly in a predefined region along the tube.

Either the at least two electrodes or the electrode array formed by the at least two electrodes extend axially in the predefined region, or the at least two electrodes extend circularly so that they surround the tube in an annular manner and form the electrode array spaced apart from each other in the axial direction. The predefined area in the axial direction therefore corresponds to a predefined array length in the axial direction of the electrode array.

Preferably, the electrode array has a predefined array length in the axial direction of 25 mm to 50 mm.

The predefined range or the predefined array length in the axial direction ensures that even in the event of (accidental) displacement of the endotracheal tube, there is still contact with the (nerve) tissue to be stimulated or the nerve tissue to be drained.

According to a further development, the endotracheal tube comprises either at least two stimulation electrodes and at least two conduction electrodes or at least two combination electrodes. The electrode array is divided into at least two, preferably three sub-arrays. The sub-arrays are arranged such that they each have a predefined sub-array distance to their adjacent sub-array.

Preferably, the sub-arrays each comprise either the at least two stimulation electrodes and the at least two dissipation electrodes or the at least two combination electrodes.

The sub-arrays are arranged one after the other in the axial direction and each completely surround the tube in a circular manner on the outside of the tube. The sub-arrays each have the sub-array spacing to their adjacent sub-arrays. Preferably, the sub-array spacing is the same between all adjacent sub-arrays. Further preferably, the sub-array spacing of the sub-arrays to each other is greater than the electrode spacing of the electrodes within a sub-array to each other. Particularly preferably, the sub-array spacing is 2 mm to 7 mm.

The electrode array is divided into at least two sub-arrays. The sub-arrays can each be formed by or comprise the same or different electrodes.

Generally, each sub-array can be formed by at least two stimulation electrodes and additionally or alternatively by at least two conduction electrodes and additionally or alternatively by at least two combination electrodes. In this respect, the stimulation electrodes or the combination electrodes comprised by a sub-array can form at least one stimulation pole and the conduction electrodes or the combination electrodes comprised by a sub-array can form at least one conduction pole. Each stimulation electrode, or derivative electrode, or combination electrode, respectively, can be jointly comprised by one or more sub-arrays. Each sub-array can comprise one or more stimulation poles and, additionally or alternatively, conduction poles.

For example, a first sub-array can be formed by stimulation electrodes only and a second sub-array can be formed by conduction electrodes only. Further, a first sub-array can comprise two stimulation electrodes forming a first stimulation pole and two conduction electrodes forming a first derivative pole, and a second sub-array can comprise two stimulation electrodes forming a second stimulation pole and two conduction electrodes forming a second derivative pole. Further, a first sub-array can comprise four combination electrodes forming a first stimulation pole and a first derivative pole, and a second sub-array can comprise four combination electrodes forming a second stimulation pole and a second derivative pole.

For example, the electrode array can be formed of four conduction electrodes and four stimulation electrodes. In this regard, the electrode array can comprise two sub-arrays. The two sub-arrays can each comprise all of the electrodes. Alternatively, one of the two sub-arrays can comprise two of the four stimulation electrodes and two of the four conduction electrodes and the other of the two sub-arrays can comprise the respective other stimulation electrodes and conduction electrodes. Alternatively, one of the two sub-arrays can comprise the four stimulation electrodes and the other of the two sub-arrays can comprise the four conduction electrodes.

Particularly preferably, the electrode array can be formed of four stimulation electrodes and four conduction electrodes or alternatively eight combination electrodes. Further, the electrode array can comprise three sub-arrays each comprising the four stimulation electrodes and four conduction electrodes or alternatively the eight combination electrodes.

By dividing the electrode array into several sub-arrays, a stimulation area in which stimulation is performed or a conduction area in which conduction is performed can be particularly precisely configured for the target tissue.

According to a further development of the present invention, at least one of the at least two electrodes is wave-shaped, sawtooth-shaped, polygon-shaped or round.

The at least one electrode extends along the tube outer surface in a wave-shaped or sawtooth shape, or the one of the at least two electrodes is polygonal (polygonal) or round, in particular rectangular or circular or elliptical, shaped on the tube outer surface. The shape is not dependent on the function of the electrode. Uniformity of the electrode shape, for example within an electrode array or among the stimulation electrodes or conduction electrodes or combination electrodes, does not necessarily have to be present.

The wave shape, sawtooth shape, polygonal or round shape of the at least one electrode enables particularly good surface coverage on the target tissue, so that the target tissue can be stimulated particularly well or stimulus responses can be derived therefrom.

According to a further development of the present invention, the lining comprises a foam structure and additionally or alternatively a spring element and additionally or alternatively an elastic balloon filled with a gas or gas mixture.

The foam structure can comprise a polymeric material having closed pores and additionally or alternatively open pores. The polymeric material with the pores exhibits elastic deformability, in particular in the radial direction towards the tube, so that it deforms when force is applied, in particular in the radial direction towards the tube, and returns to its original shape after the force is removed.

The spring element is formed to deform elastically under the influence of force, especially in the radial direction on the tube, and to return to its original shape after the force is removed.

The balloon filled with gas (e.g. nitrogen, inert gas, etc.) or a gas mixture (e.g. air) has an elastic envelope. Due to the elasticity of the envelope, the balloon can be elastically deformed under the effect of force, in particular in the radial direction on the tube, whereby the balloon returns to its original shape after the force is removed.

The foam structure, the spring element and the balloon filled with gas or a gas mixture offer a high elastic deformability, whereby the electrode(s) arranged thereon can adapt particularly well to different anatomies.

According to a further development of the present invention, the tube comprises polyvinyl chloride, PVC, preferably reinforced PVC, as the material.

The endotracheal tube can comprise a tube made at least partly of PVC and preferably of transparent PVC and particularly preferably of reinforced PVC (double-layer PVC with metal reinforcement, e.g. spiral wires).

PVC is a soft, flexible and tissue-compatible material that has a low allergy potential. In addition, PVC can be sterilised by steam, y-radiation and gassing with ethylene oxide (EO, C2H4O).

According to a further embodiment of the present invention, a distal end of the tube tapers at an acute angle, preferably at an angle between 30 and 45° and more preferably at an angle of 38°.

The angle at which the distal end of the tube converges ensures easier insertion into the trachea.

According to a further development of the present invention, at least one of the electrodes or at least one of the paddings with the corresponding at least one electrode is fully integrated into the tube. Additionally or alternatively, at least one of the electrodes or at least one of the paddings with the corresponding at least one electrode is formed as an adhesive electrode and is attached to the outside of the tube. Additionally or alternatively, at least one of the electrode arrays is formed as an electrode sleeve and is attached tangentially around the tube outer surface.

The fully integrated electrode or lining with electrode(s) are arranged directly on the outside of the tube and are firmly (non-detachably) mechanically connected to the tube (e.g. by screen printing, dispensing, piezo-jetting or thermobonding).

The endotracheal tube with fully integrated electrode can be used immediately without prior attachment and positioning steps.

The adhesive electrode can be used universally and can be detachably attached (e.g. adhesive bond) to the tube at the appropriate point on the outside of the tube shortly before the surgical procedure. The adhesive electrode is sufficiently firmly mechanically connected to the outside of the tube so that the adhesive electrode cannot detach from the tube during the surgical procedure.

The electrode cuff can be used universally and can be detachably attached (e.g. adhesive connection) in a circular manner around the tube just before the surgical intervention at the corresponding point on the outside of the tube. The electrode cuff is sufficiently firmly mechanically connected to the outside of the tube so that the electrode cuff cannot detach from the tube during the surgical procedure.

Neither the adhesive electrode nor the electrode cuff need to be processed, e.g. cut, before attachment, as they adapt to the shape of the tube. Suitable electrodes can therefore be selected according to the patient and attached quickly and easily before the surgical procedure.

According to a further embodiment of the present invention, the endotracheal tube comprises four to 20, preferably eight to eleven electrodes.

Due to the electrodes arranged in a circular distribution in the electrode array around the tube, tissue can be stimulated consistently well even when the endotracheal tube is rotated during the surgical procedure, and stimulus responses can additionally or alternatively be derived consistently well.

According to a further development, at least one of the electrodes is at least partially made of conductive material, preferably silver, gold, platinum or carbon-based printing paste.

The conductive material allows an electrical stimulation signal to be induced from the IMO system to the patient's tissue by means of the electrode made of the conductive material or to transmit stimulation responses from nerve tissue to the IOM system by means of the electrode made of the conductive material.

Silver, gold and platinum are particularly suitable materials for the electrode because they have good electrical conductivity on the one hand and good biocompatibility on the other. In addition, they can be applied in thin layers to avoid sharp edges in the surface profile and prevent injuries.

Carbonised printing paste as a material for the electrode also has good electrical conductivity (in the processed state) and is particularly easy to process into an electrode.

According to a further development of the present invention, the at least one adhesive electrode and additionally or alternatively the at least one electrode sleeve comprise a distance indicator. The distance indicator is formed to indicate the optimal distance of the at least one adhesive electrode and additionally or alternatively of the at least one electrode cuff to the distal end of the tube (tube tip) or to the fixation element (inflatable element/ balloon).

The distance indicator can be shaped as a protrusion or tab, whereby the length of the protrusion or tab corresponds to the optimal distance of the at least one adhesive electrode and additionally or alternatively of the at least one electrode sleeve to the tube tip or fixation element. Thus, the optimal distance of the electrode or electrode sleeve to the tip of the tube or to the fixation element is achieved when the tab is in contact with the tip of the tube or the fixation element.

The distance indicator enables a particularly simple and yet precise attachment of adhesive electrodes or electrode sleeves to the outside of the tube.

According to a further embodiment of the present invention, the endotracheal tube further comprises an X-ray contrast strip. The X-ray contrast strip is arranged along the tube. The X-ray contrast strip is configured to visibly display a position of the tube in a trachea of a patient, in particular in an advancement direction of the endotracheal tube, in an X-ray image.

The X-ray contrast strip can extend substantially in an axial direction along the entire tube. The X-ray contrast strip can be integrated or glued into the tube. In addition, the X-ray contrast strip can have a scale that is visible on an X-ray image and that allows better determination of the position and orientation of the endotracheal tube in the X-ray image.

By means of the X-ray contrast strip, the position of the endotracheal tube in the trachea of a patient can be quickly and accurately checked intra-operatively by X-ray inspection.

According to a further embodiment of the present invention, the endotracheal tube further comprises at least one pressure sensor configured to measure muscle contractions in a trachea of a patient.

The pressure sensor can be formed as a ring arranged circularly over 360° around the outside of the endotracheal tube. Alternatively, several individual pressure sensors can be arranged circularly over 360° or tangentially distributed on the tube outer side around the endotracheal tube. A 360° sensor coverage has the advantage of rotational stability of the measured signal.

The pressure sensor(s) can be made of a thin membrane and in particular can be strain gauges that pick up the mechanical strain and/or compression caused by pressure (muscle contractions in a trachea) and convert it into electrical signals. At the same time, the electrical resistance changes. Depending on the resistance, the user of the endotracheal tube can recognise whether the tube is correctly positioned in the trachea or not, thus enabling position control.

To avoid rejection reactions, the sensor(s) is/are biocompatible coated or at least made of a biocompatible material. Furthermore, the sensors are firmly integrated in the tube or removable (see adhesive electrodes, cuff). The sensor or the individual sensors can have any shape (round, polygonal, wave-shaped, saw-tooth-shaped, etc.). The pressure sensors can lie on an underpadding analogous to the electrodes.

The pressure sensor(s) provide a second physiological recording in addition to the EMG recording, which can be used to monitor the nerve or muscle tissue during intra-operative neuromonitoring. The pressure measurement is also not susceptible to artefacts and thus enables a virtually artefact-free derivation.

According to a further development, the endotracheal tube further comprises an optical system. The optics is formed to visually check the position of the endotracheal tube in a trachea. The optic is further configured to visually check the vocal cords and their movement, in particular during insertion and/or removal of the endotracheal tube into/from the patient's trachea.

The optics comprises at least two light guide bundles, wherein at least one light guide bundle feeds light from a light source into the surgical field and at least one other light guide bundle picks up an image (of the surgical field) and forwards it to a processing unit for displaying the image on a monitor.

The optics are preferably located on an inner side of the tube of the endotracheal tube or alternatively on the tube outer side. Attaching the optics to the inside has the advantage that the geometry (outer diameter) of the endotracheal tube does not change. The advantage of placing the optics on the outside is that the inner diameter and thus the volume through which the patient is ventilated is not reduced.

The light guide bundles can be connected at the proximal end of the endotracheal tube by means of an adapter to an overall system such as an IOM system, whereby this has a special video input and can display the image on a monitor. Furthermore, the overall system/ IOM system can comprise the light source.

According to a further embodiment, the endotracheal tube according to one of the preceding claims further comprises an intubation depth marker. The depth marker is arranged along the tube. The depth marker is configured to indicate the distance to the tip of the tube in millimetres [mm] and thus to indicate the intubation depth of the tube in a trachea of a patient in the advancement direction of the endotracheal tube.

The depth marker can optionally additionally have two distinct markings above the inflatable element in the vicinity of the optional pressure sensors, which also rest against the vocal folds. These additional markings are in the form of radiopaque strips and indicate the optimum position of the endotracheal tube relative to the vocal folds under X-ray control.

According to a further development of the present invention, the at least one counter electrode is formed as a needle electrode or as a surface electrode. The at least one counter electrode is further configured to stimulate tissue subcutaneously or transcutaneously in combination with the at least one stimulation electrode or the at least one combination electrode.

The at least one counter electrode forms a counter pole to the stimulation pole(s). Therefore, the at least one counter electrode does not have to be attached as close to the tissue to be stimulated as the at least one stimulation electrode/combination electrode. Thus, the counter pole can be implemented in a particularly simple manner by a needle electrode which is pierced into the patient's skin in the vicinity of the tissue to be stimulated and enables subcutaneous stimulation, or by a surface electrode which is placed (glued or sucked) on the patient's skin in the vicinity of the tissue to be stimulated and enables transcutaneous stimulation.

According to a further embodiment of the present invention, electrical stimulation is performed in a monopolar fashion by means of the at least one stimulation electrode or the at least one combination electrode of the endotracheal tube and the at least one counter electrode of the system. Alternatively, electrical stimulation is bipolar by means of at least two stimulation electrodes and/or combination electrodes of the endotracheal tube. Alternatively, electrical stimulation is performed multipolar by means of multiple stimulation electrodes and/or combination electrodes of the endotracheal tube.

In monopolar stimulation, the stimulation is carried out between a stimulation electrode (at least one stimulation/combination electrode) and a reference electrode (at least one counter electrode). Thus, the stimulation poles are further apart. This serves to first obtain a rough overview of the surgical field and to be able to estimate where a nerve is located. The current spread is relatively large; the current spreads homogeneously in the tissue and stimulates nerves through the tissue. The penetration depth of the stimulation current is approximately proportional to the stimulation intensity.

With bipolar stimulation, the stimulation is carried out between two poles that are close to each other (at least two stimulation/combination electrodes). Selective stimulation is used when a nerve is visible and the user wants to examine it functionally or to determine which nerve is involved. Bipolar stimulation is less susceptible to interference than monopolar stimulation because there is a focused current spread between the electrode contacts.

With multipolar stimulation, the stimulation is carried out simultaneously by means of several stimulation contacts (multiple stimulation/combination electrodes). The stimulation of the nerves is carried out over a larger area than with bipolar stimulation. By complex arrangement of the multiple stimulation contacts (multiple stimulation/combination electrodes), the field propagation can be specifically influenced.

The above aspects, embodiments and further developments can be combined with each other as desired, if this makes sense. Further possible embodiments, further developments and implementations of the invention also include combinations of features of the invention described above or below with respect to the embodiments that are not explicitly mentioned. In particular, the skilled person will thereby also add individual aspects as improvements or additions to the respective basic form of the present invention.

### BRIEF DESCRIPTION OF THE DRAWINGS

The present invention is explained in more detail below with reference to the examplary embodiments shown in the schematic figures, wherein:

FIGS. 1a to 1d represent four embodiments of the endotracheal tube according to the first aspect of the present invention;

FIGS. 2a to 2b represent arrangements of electrodes in an electrode array or sub-array;

FIGS. 3a to 3c represent arrangements of circularly extending stimulation electrodes, conduction electrodes and combination electrodes in sub-arrays;

FIGS. 4a to 4c represent arrangements of axially extending stimulation electrodes, conduction electrodes and combination electrodes in sub-arrays;

FIG. 5 represents an embodiment of the endotracheal tube according to the second aspect of the present invention;

FIGS. 6a to 6b represent sectional views of the exemplary embodiment of the endotracheal tube according to the second aspect of the present invention;

FIGS. 7a to 7c represent various paddings with electrodes arranged thereon;

FIG. 8 represents an adhesive electrode;

FIG. 9 represents an exemplary embodiment of an endotracheal tube according to the first or the second aspect of the present invention with X-ray contrast strips;

FIG. 10 represents an embodiment of an endotracheal tube according to the first or the second aspect of the present invention including depth marking;

FIG. 11 represents an embodiment of an endotracheal tube according to the first or the second aspect of the present invention including integrated optics;

FIGS. 12a to 12c represent three embodiments of an endotracheal tube according to the first or the second aspect of the present invention with pressure sensors;

FIG. 13 represents an exemplary embodiment of a system according to the third aspect of the present invention;

FIG. 14 represents the method for deriving stimulus responses during intra-operative neuromonitoring according to the fourth aspect of the present invention;

FIG. 15 represents a schematic representation of an overall system;

FIG. 16 represents the method for classifying tissue types according to the fifth aspect of the present invention;

FIG. 17 represents an embodiment of the endotracheal tube according to the first or second aspect of the present invention, which is configured to perform the method for classifying.

The accompanying figures of the drawing are intended to provide a further understanding of embodiments of the invention. They illustrate embodiments and, in connection with the description, serve to explain principles and concepts of the invention. Other embodiments and many of the advantages mentioned will be apparent with reference to the drawings. The elements of the drawings are not necessarily shown to scale with respect to each other.

In the figures of the drawing, identical elements, features and components which have the same function and the same effect are each given the same reference signs, unless otherwise stated.

### Description Of Exemplary Embodiments

In FIGS. 1a to 1d, four embodiments of an endotracheal tube 1a according to the first aspect of the present invention are schematically illustrated.

The endotracheal tube la can be used in intra-operative neuromonitoring to monitor nerve and muscle activity and comprises a tube 2, a fixation element 3 and an electrode array 5.

The tube 2 has a proximal end 7 and a distal end (tube tip) 8. Over its entire length, the tube has a substantially circular cross-section with a constant inner diameter or outer diameter. The thickness of the wall of the tube 2 is very small compared to its inner diameter, which means that the inner diameter of the tube 2 is essentially equal to its outer diameter.

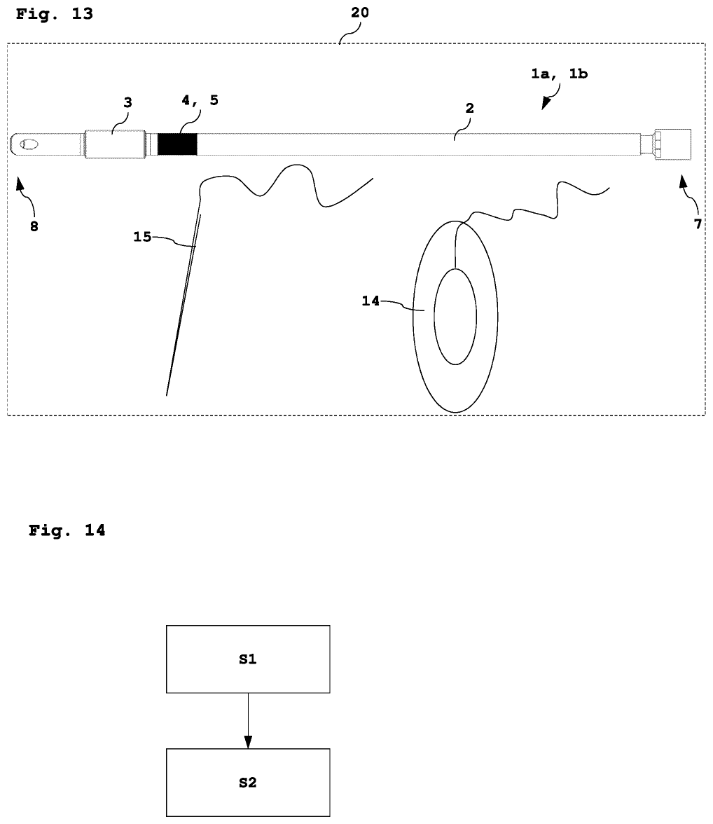

The tube 2 is made of reinforced PVC and has an opening at the proximal end 7 in the axial direction with a connector that can be connected to a ventilator (see FIG. 13) by means of a suitable tube. At the distal end 8, the tube 2 also has an opening in the axial direction and also an additional opening laterally.

At a predefined element distance from the tube tip 8, a fixation element 3 in the form of an inflatable balloon is located on a tube outer side of the tube 2. The balloon is made of silicone and can be inflated by means of a tube running along an inner side of the tube 2 with a Luer connection at its proximal end by means of a disposable syringe.

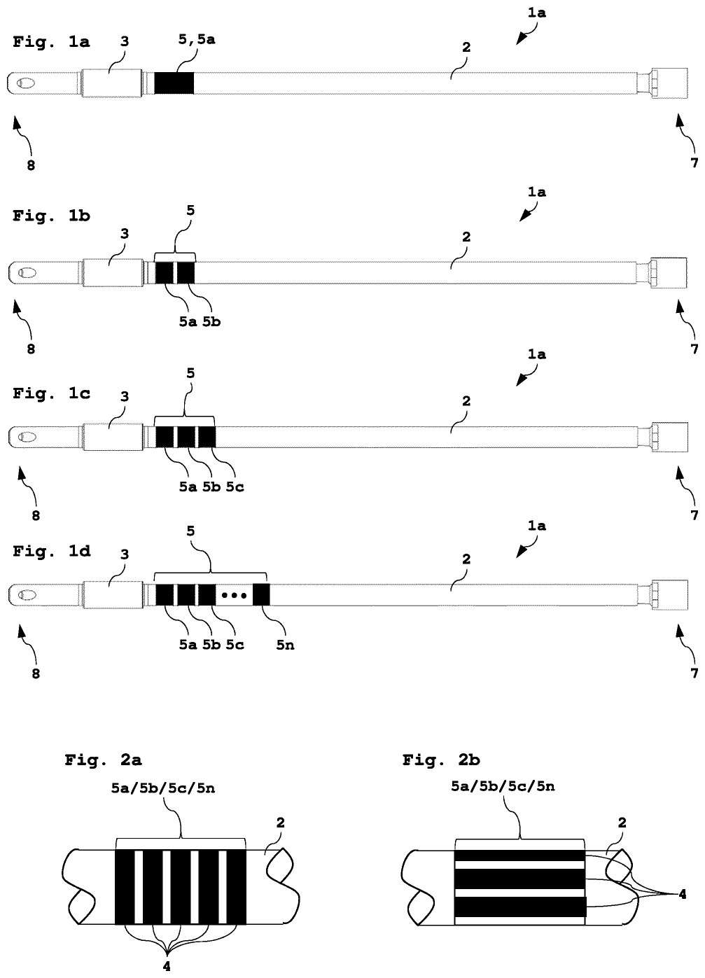

Further proximal than the balloon 3, an electrode array 5 is arranged on the tube exterior at a predefined array distance from the tube tip 8. The electrode array 5 comprises at least two electrodes (see FIGS. 2a and 2b), which can be formed as stimulation electrodes and/or conduction electrodes and/or combination electrodes. The electrodes can be connected to an IOM system by means of a suitable connector (see FIG. 13). The electrode array 5 extends in the axial direction over a predefined array length and completely surrounds the tube 2 in a circular manner. On the outside of the tube, the at least two electrodes are distributed over 360° around the tube 2 in the electrode array 5. The electrodes can extend in an axial direction or circularly (annularly) along the tube 2 within the electrode array (see FIGS. 2a and 2b).

The electrode array 5 can be divided into several sub-arrays 5a, 5b, 5c, 5n. The exemplary embodiment shown in FIG. 1a has an electrode array (5) without subdivision or with only one sub-array 5a. The exemplary embodiment shown in FIG. 1b has an electrode array 5 divided into two sub-arrays 5a, 5b. The preferred exemplary embodiment shown in FIG. 1c has an electrode array 5 divided into three sub-arrays 5a, 5b, 5c. The exemplary embodiment shown in FIG. 1d has an electrode array 5 divided into four or more sub-arrays 5a, 5b, 5c, . . . , 5n. Each sub-array 5a, 5b, 5c, 5n fully comprises at least two of the electrodes of the electrode array 5. In other words, at least two electrodes of the electrode array 5 each form a sub-array 5a, 5b, 5c, 5n. The sub-arrays 5a, 5b, 5c, 5n are each arranged at a predefined sub-array spacing from each other in the axial direction.

The endotracheal tube 1a is inserted into the trachea of a patient during a surgical procedure such as a thyroid operation and is advanced sufficiently to allow the patient to be ventilated under anaesthesia by a ventilator during the surgical procedure. The balloon 3 is inflated once the endotracheal tube 1a is correctly advanced and positioned to ventilate the patient. This fixes the endotracheal tube 1a or the tube 2 in the trachea so that the endotracheal tube 1a can be prevented from moving or twisting during the surgical procedure. The electrode array 5 or the electrodes 4 are arranged at a predefined array distance on the outside of the tube in such a way that the electrodes 4 are in contact with the vocal folds when the endotracheal tube 1a is correctly advanced and positioned to ventilate the patient and is fixed in place by means of the inflated balloon 3. Muscle tissue can be stimulated on the vocal folds by means of the electrodes 4 during the surgical procedure and, additionally or alternatively, stimulus responses can be derived from the stimulated muscle tissue and thus monitored by means of intra-operative neuromonitoring of the NLR, particularly during thyroid surgery.

In FIGS. 2a and 2b, a sub-array 5a/5b/5c/5n or an electrode array, if this only comprises a sub-array 5a, is shown schematically on the tube 2.

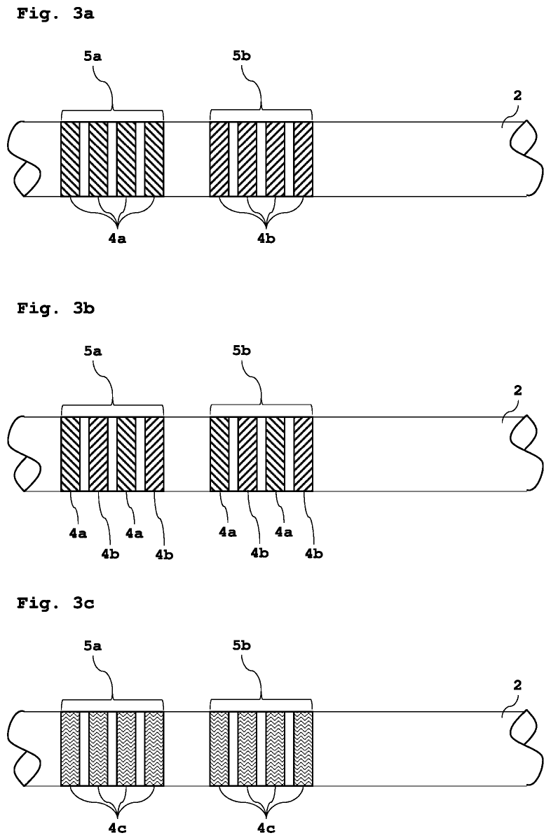

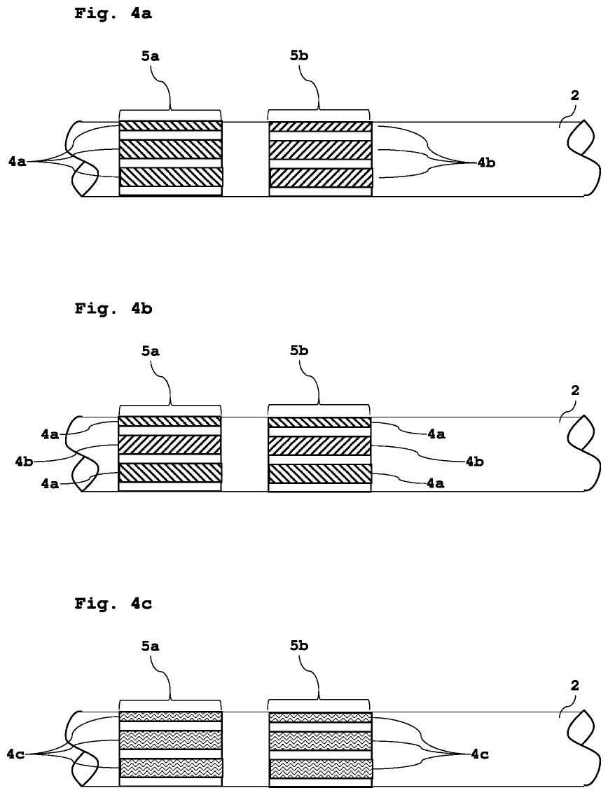

The sub-array 5a/5b/5c/5n completely comprises at least two, in this case exemplarily five, electrodes 4. The electrodes 4 are exemplarily formed here as substantially rectangular strips of conductive material. The electrodes 4 can extend in the sub-array 5a/5b/5c/5n in the axial direction, as shown in FIG. 2b, or circularly, as shown in FIG. 2a, along the tube 2. In this case, the electrodes 4 are each spaced apart from each other at an equal predefined electrode distance. The electrodes 4 can be stimulation electrodes, conduction electrodes or combination electrodes (see FIGS. 3a to 4c).

In the sub-array 5a/5b/5c/5n shown in FIG. 2a, the electrodes 4 running circularly over 360° around the tube 2 are spaced from each other in the axial direction by the electrode spacing. In the sub-array 5a/5b/5c/5n shown in FIG. 2b, the electrodes (4) running in the axial direction along the tube 2 are distributed circularly over 360° around the tube 2, and are spaced apart from each other in each case by the electrode spacing.

In FIGS. 3a to 3c, a tube 2 with two sub-arrays 5a, 5b, each comprising stimulation electrodes 4a and/or conduction electrodes 4b or combination electrodes 4c, is shown schematically.

The two sub-arrays 5a, 5b are spaced apart from one another in the axial direction at the predefined sub-array spacing and comprise here, by way of example, four electrodes 4a, 4b, 4c in each case, which extend circularly over 360° around the tube 2 and are arranged spaced apart from one another in the axial direction at the predefined electrode spacing. The sub-array spacing is greater than the electrode spacing.

Each stimulation electrode 4a or combination electrode 4c can form a stimulation pole alone or in combination with one or more further stimulation electrodes 4a or combination electrodes 4c. Thus, depending on the number ks of stimulation or combination electrodes 4a/4c, 1 to kₛ stimulation poles can be formed. By means of stimulation poles, tissue (e.g. the muscle tissue on the vocal folds) or the nerves connected to it can be stimulated by means of alternating electrical signals.

Each conduction electrode 4b or combination electrode 4c can form a stimulation pole alone or in combination with one or more other conduction electrodes 4b or combination electrodes 4c. Thus, depending on the number k_{A} of the conduction electrodes or combination electrodes 4b/4c, 1 to k_{A} conduction poles can be formed. Stimulus responses from stimulated tissue can be derived as EMGs by means of conduction poles.

In the sub-arrays 5a, 5b shown in FIG. 3a, the first sub-array 5a comprises exclusively four stimulation electrodes 4a, and the second sub-array comprises exclusively four conduction electrodes 4b. Thus, the stimulation pole(s) are clearly spatially separated from the conduction poles, resulting in an improved signal-to-noise ratio (SNR).

In the sub-arrays 5a, 5b shown in FIG. 3b, the first sub-array 5a and the second sub-array 5b each comprise two stimulation electrodes 4a and two conduction electrodes 4b. The stimulation electrodes 4a and the conduction electrodes 4b are arranged alternately in the two sub-arrays 5a, 5b, in this case as an example. In particular, if each stimulation electrode forms a separate stimulation pole and each conduction electrode forms a separate conduction pole, the tissue to which the electrodes are applied can be monitored with high spatial resolution.

In the sub-arrays 5a, 5b shown in FIG. 3b, the first sub-array 5a and the second sub-array 5b each comprise four combination electrodes 4c. The combination electrodes can each form a stimulation pole for stimulating tissue, and can then form a discharge pole for discharging stimulus responses from the stimulated tissue. Depending on how the combination electrodes are connected, EMGs with high SNR or high spatial resolution can be derived.

FIGS. 4a to 4c each schematically show a tube 2 with, by way of example, two sub-arrays 5a, 5b, each comprising stimulation electrodes 4a and/or conduction electrodes 4b or combination electrodes 4c. The sub-arrays 5a, 5b shown in FIGS. 4a to 4c differ from those shown in FIGS. 3a to 3c essentially only in the course of the electrodes 4a, 4b, 4c within the sub-arrays 5a, 5b. Therefore, only the differences with respect to FIGS. 3a to 3c are explained below and otherwise reference is made with respect to the explanations given with respect to FIGS. 3a to 3c.

The two sub-arrays 5a, 5b are spaced apart from each other in the axial direction at the predefined sub-array spacing and comprise here, by way of example, in each case several electrodes 4a, 4b, 4c (three each visibly shown) which extend in the axial direction and are arranged distributed over 360° and spaced apart from each other around the tube 2 at the predefined electrode spacing. The sub-array spacing is greater than the electrode spacing.

In the case of the sub-arrays 5a, 5b shown in FIG. 4a, the first sub-array 5a comprises exclusively stimulation electrodes 4a and the second sub-array comprises exclusively conduction electrodes 4b. Thus, the stimulation pole(s) are clearly spatially separated from the conduction poles, resulting in an improved signal-to-noise ratio (SNR).

In the sub-arrays 5a, 5b shown in FIG. 4b, the first sub-array 5a and the second sub-array 5b each comprise a plurality of stimulation electrodes 4a and a plurality of conduction electrodes 4b. The stimulation electrodes 4a and the conduction electrodes 4b are arranged alternately in the two sub-arrays 5a, 5b as an example. Here, in the second sub-array 5b, stimulation electrodes 4a are also opposite the stimulation electrodes 4a in the first sub-array 5a. The same applies to the conduction electrodes 4b. The electrodes of the second sub-array 2b can also be circularly displaced with respect to those of the first sub-array 5a. Stimulation electrodes 4a in the first sub-array 5a can also be opposed by conduction electrodes 4b in the second sub-array 5b, and likewise conduction electrodes 4b in the first sub-array 5a can be opposed by stimulation electrodes 4a in the second sub-array 5b. In particular, if each stimulation electrode forms a separate stimulation pole and each conduction electrode forms a separate conduction pole, the tissue to which the electrodes are applied can be monitored with high spatial resolution.

In the sub-arrays 5a, 5b shown in FIG. 4b, the first sub-array 5a and the second sub-array 5b each comprise a plurality of combination electrodes 4c. The combination electrodes can each form a stimulation pole for stimulating tissue, and can then form a discharge pole for discharging stimulus responses from the stimulated tissue. Depending on how the combination electrodes are connected, EMGs with high SNR or high spatial resolution can be derived.

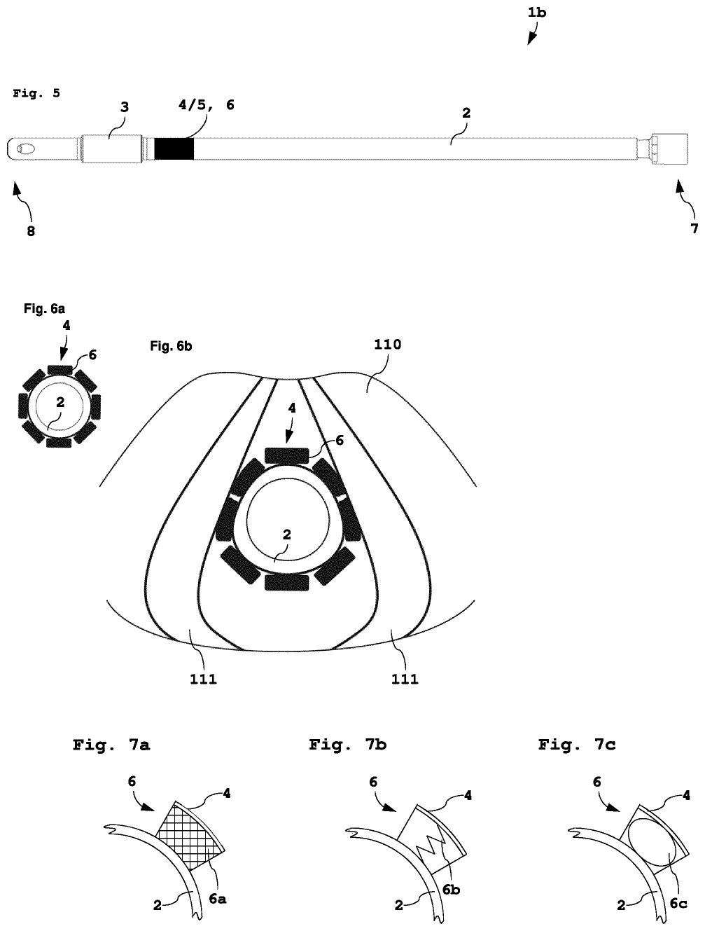

FIG. 5 schematically illustrates an embodiment of an endotracheal tube 1b according to the second aspect of the present invention. The endotracheal tube 1b shown in FIG. 5 differs from the endotracheal tube 1a of FIGS. 1a to 4c with its electrode array comprising at least two electrodes essentially in that at least one electrode 4 or electrode array such as that of FIGS. 1a to 4c is included, and in that a padding 6 is associated therewith. Therefore, only the differences to the endotracheal tube 1a of FIGS. 1a to 4c will be discussed below, wherein reference is otherwise made with respect to the explanations of FIGS. 1a to 4c.

The at least one electrode 4 or at least one electrode of the electrode array 5 is supported by the padding 6 on the tube outer side of the tube 2. For this purpose, the padding 6 is arranged on the tube outer side and the electrode(s) 4 is/are applied to the padding 6. The padding 6 has an elastic deformability (elasticity) at least in the radial direction towards the tube 2. When force is applied to the padded electrode(s) 4 in the radial direction towards the tube 2, the padding 6 deforms elastically towards the tube 6 so that the padded electrode(s) 4 is/are displaced towards the tube 2. As soon as the force on the electrode(s) 4 decreases or ceases, the padding 6 elastically deforms back so that the electrode(s) 4 are moved away from the tube 2 to their original position.

The padding 6 can be formed separately for each electrode 4 (see FIGS. 6a and 7a to 7c), or can be formed essentially annularly/circularly around the tube 2.

When the endotracheal tube 1b is inserted into the trachea, the padding 6 is compressed by the wall of the trachea. The at least one padded electrode 4 or the at least two padded electrodes of the electrode array 5 are pressed against the trachea or the vocal folds by the padding 6 (see FIG. 6b). The padded electrode(s) thus nestle(s) optimally against the tissue to be stimulated (e.g. muscle tissue). At the same time, the electrodes are prevented from moving by the padding 6. This means that the tissue can be stimulated particularly well and stimulus responses can be derived from it.

In FIGS. 6a and 6b the endotracheal tube 1b is shown schematically in section through the at least one electrode 4 or the electrode array 5 and through the padding 6.

In this case the padding 6 is divided into eight segments. Each segment of the padding 6 supports an electrode 4, for example of the electrode array, on the tube 2.

If the endotracheal tube 1a is inserted into a trachea 110, the padding 6 or its segments are elastically deformed towards the tube 2 at the points where electrodes 4 rest against the wall of the trachea 110 or the vocal folds 111. Due to the elasticity of the padding 6, it exerts a counterforce on the corresponding electrodes 4 so that they are pressed against the wall of the trachea 110 or the vocal folds 111.

FIGS. 7a to 7c schematically show three examples of paddings 6 or their segments.

The padding 6 shown in FIG. 7a has an elastic foam structure 6a which elastically supports the electrode 4 relative to the tube 2.

The padding 6 shown in FIG. 7b has an elastic spring element 6b which elastically supports the electrode 4 relative to the tube 2.

The padding 6 shown in FIG. 7c has an elastic balloon 6c filled with a gas or gas mixture, which elastically supports the electrode 4 relative to the tube 2.

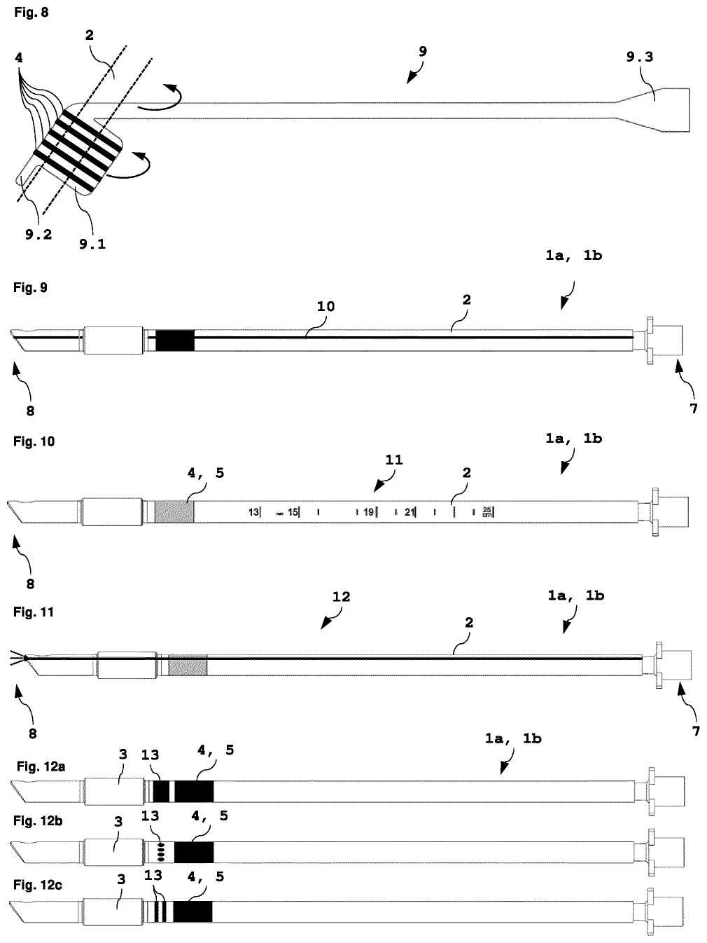

In FIG. 8 an adhesive electrode 9 is shown schematically. The adhesive electrode 9 comprises an electrode section 9.1, a distance indicator 9.2 and an electrode connection 9.3.

The electrode section has at least one electrode 4, here exemplarily five electrodes 4. The electrode section 9.1 can also have a sub-array or an electrode array with one or more sub-arrays.

The distance indicator 9.2 is formed on the electrode section 9.1 and indicates the optimum distance or array distance to the tube tip/distal end or fixation element of the endotracheal tube of FIGS. 1a to d or 5.

The electrode connector is formed on a proximal end of the adhesive electrode 9 and is used to connect to an IOM system.

The adhesive electrode 9 is first optimally positioned on a tube 2 by means of the distance indicator 9.2 (e.g., at the predefined array distance), and is then attached around the tube 2 and fixed, for example, by means of an adhesive. The endotracheal tube with the adhesive electrode 9 can then be inserted into the trachea and connected to an IOM system by means of the electrode connection 9.3.

In FIG. 9, an exemplary embodiment of an endotracheal tube 1a, 1b according to the first or second aspect of the present invention is shown schematically. The endotracheal tube 1a, 1b comprises an X-ray contrast strip 10.

The X-ray contrast strip 10 is radiopaque, and extends in an axial direction from the tip 8 of the tube to the proximal end 7 of the endotracheal tube 1a, 1b. The X-ray contrast strip can be arranged on the outside of the tube, the inside of the tube 2 or in the wall of the tube 2.

By means of the X-ray contrast strip 10, the position of the endotracheal tube 1a, 1b in the trachea can be checked under X-ray control.

In FIG. 10, an exemplary embodiment of an endotracheal tube 1a, 1b according to the first or second aspect of the present invention is schematically shown. The endotracheal tube 1a, 1b has a depth marker 11 for intubation depth.

The depth marker 11 has a plurality of depth indications on the tube outer surface indicating the distance from the tube tip 8 in millimetres, and extends in axial direction.

By means of the depth marker 11, the intubation depth of the endotracheal tube 1a, 1b in the trachea and the position of the electrode(s) relative to the vocal folds can be checked.

FIG. 11 schematically illustrates an exemplary embodiment of an endotracheal tube 1a, 1b according to the first or second aspect of the present invention. The endotracheal tube 1a, 1b comprises an integrated optic 12.

The optics 12 comprises at least two light guide bundles. At least one of the light guide bundles guides light from a light source at the tip of the tube 8 into the surgical field. At least one other of the light guide bundles transmits an image (of the surgical field) from the tube tip 8 to a processing unit for displaying the image on a monitor. For this purpose, the light guide bundles preferably run on an inner side of the tube 2, or alternatively run on the tube outer side. The light guide bundles can be connected at the proximal end 7 of the endotracheal tube 1a, 1b by means of an adapter to an overall system such as an IOM system, whereby this also comprises the light source in addition to the monitor.

Thus, by means of the optics 12, it is possible to visually check the position of the endotracheal tube 1a, 1b in a trachea, and to visually check the vocal cords as well as their movement, in particular during insertion and/or removal of the endotracheal tube 1a, 1b into/from the patient's trachea.

In FIGS. 12a to 12c, three embodiments of an endotracheal tube 1a, 1b according to the first or second aspect of the present invention are schematically shown. The endotracheal tube 1a, 1b comprises at least one pressure sensor 13.

The at least one pressure sensor 13 can be formed as a ring as is shown in FIG. 12a, or as two rings as is shown in FIG. 12c. The at least one ring-shaped pressure sensor 13 is arranged circularly over 360° on the tube exterior around the endotracheal tube 1a, 1b. Alternatively, several individual pressure sensors can be arranged circularly over 360° or tangentially distributed on the tube outer side around the endotracheal tube 1a, 1b as shown in FIG. 12b. The pressure sensor(s) 13 are (is) arranged between the fixation element 3 and the at least one electrode 4 (of the electrode array 5).

The pressure sensor or the pressure sensors 13 can consist of a thin membrane, and can in particular be strain gauges which record the mechanical strain and/or compression caused by pressure (muscle contractions in a trachea), and convert the mechanical strain and/or compression into electrical signals.

The pressure sensor(s) 13 allow, in addition to EMG conduction by means of the electrodes 4 (of the electrode array 5), a second physiological derivation that can be used to monitor the nerve or muscle tissue during intra-operative neuromonitoring. Furthermore, the pressure measurement is not susceptible to artefacts, and thus enables an almost artefact-free derivation.

FIG. 13 schematically illustrates an exemplary embodiment of the system 20 according to the third aspect of the present invention. The system 20 comprises the endotracheal tube 1a, 1b of FIGS. 1a to 12c, and at least one separate conduction electrode 14, and at least one counter electrode 15. At least one electrode 4 (of the electrode array 5) is formed as a stimulation electrode 4a or as a combination electrode 4c.

The at least one separate conduction electrode 14 is formed as a surface electrode, and can be attached to the patient in the vicinity of the stimulated tissue (e.g. on the outside of the patient's neck). The separate conduction electrode 14 can be used to derive stimulus responses from the stimulated tissue instead of or in addition to the conduction electrode(s) 4b or combination electrode(s) 4c of the endotracheal tube 1a, 1b on the tube outer side of the stimulated nerve tissue. The conduction of the stimulus responses thus is carried out optionally by means of at least one of the conduction electrodes 4b/combination electrodes 4c of the endotracheal tube 1a, 1b, and additionally or alternatively by means of the at least one separate conduction electrode 14.

The optional at least one counter electrode 15 is formed as a needle electrode, and can be attached externally to the patient. With the optional at least one counter electrode 15 and with the at least one stimulation electrode 4a or the at least one combination electrode 4c, the tissue can be stimulated in a monopolar fashion. For this purpose, the at least one counter electrode 15 is attached to the patient at a different location than the stimulation electrode(s) 4a or combination electrode(s) 4c of the endotracheal tube 1a, 1b, and serves as a counter pole to the stimulation pole(s) formed by the stimulation electrode(s) 4a/ combination electrode(s) 4c. In particular, by means of a stimulation electrode or combination electrode 4a/4c of the endotracheal tube 1a, 1b on its tube outer side (a stimulation pole) and a 15 counter electrode (counter pole), the (nerve) tissue can be stimulated in a monopolar fashion.

Alternatively, the counter electrode 15 can be formed by a stimulation electrode 4a or combination electrode 4c located on the outside of the tube, or alternatively can be formed by an interconnection of several of the electrodes on the outside of the tube. The monopolar stimulation of (nerve) tissue is performed accordingly by means of the counter electrode 15 on the outside of the tube and a further stimulation electrode 4a or combination electrode 4c.

In FIG. 14, the method for deriving stimulus responses during intra-operative neuromonitoring according to the fourth aspect of the present invention is schematically illustrated. The method for deriving stimulus responses during intra-operative neuromonitoring comprises the steps of electrical stimulation S1 and derivation S2, and is performed using the endotracheal tube 1a, 1b of FIGS. 1a to 12c or the system 20 shown in FIG. 13.

In step S1, tissue (e.g. muscle tissue) is electrically stimulated. The step of electrically stimulating is performed by means of at least one stimulation electrode 4a or at least one combination electrode 4c of the endotracheal tube 1a, 1b, and optionally additionally by means of at least one counter electrode 15 of the system 20. The step of electrically stimulating can be performed in a monopolar fashion by means of the at least one stimulation electrode 4a/combination electrode 4c and the at least one counter electrode 15. Further, the step of electrically stimulating can be bipolar by means of at least two stimulation electrodes 4a/combination electrodes 4c. Furthermore, the step of electrically stimulating can be multipolar by means of multiple stimulation electrodes 4a/combination electrodes 4c.

In step S2, a stimulus response of the stimulated tissue (e.g. at the vocal folds) is derived. The deriving is carried out by means of at least one conduction electrode 4b, or at least one combination electrode 4c of the endotracheal tube 1a, 1b, or by means of at least one separate conduction electrode 14 of the system 20.

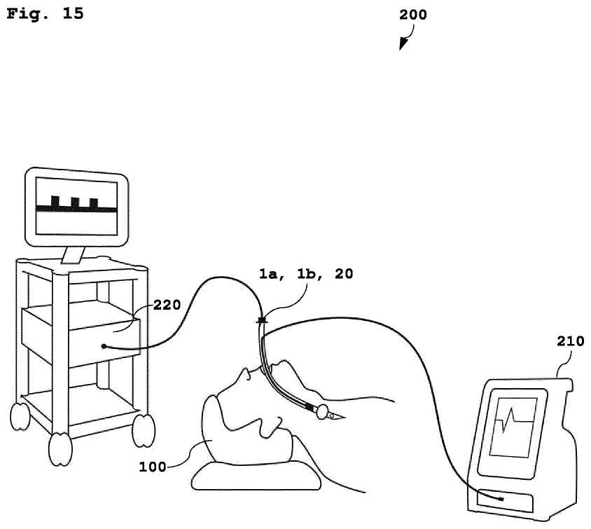

In FIG. 15, an overall system 200 is shown schematically. The complete system 200 comprises the endotracheal tube 1a, 1b of FIGS. 1a to 12c, or the system 20 shown in FIG. 13 as well as an IOM system 210 and a ventilator 220.

The tube of the endotracheal tube 1a, 1b is fluidly connected to the ventilator 220 by means of a suitable tube connected to the proximal end of the tube. The electrodes of the endotracheal tube 1a, 1b and, if present, the separate conduction electrode and optional counter electrode of the system 20 are electrically connected to the IOM system 210 by means of one or more suitable connecting cables.

The endotracheal tube 1a, 1b is inserted into the trachea of a patient 100 during a surgical procedure, and fixed there by means of its balloon. During the surgical procedure, the patient is ventilated with air by the ventilator 220 by means of the tube of the endotracheal tube 1a, 1b, and is thus supplied with oxygen. For intra-operative monitoring of the muscle and nerve tissue in the area to be operated, the muscle tissue at the vocal folds is stimulated by means of the stimulation electrode(s)/combination electrode(s) of the endotracheal tube 1a, 1b and optionally the counter electrode(s) of the system 20 in a monopolar, bipolar or multipolar fashion by delivering an appropriate alternating electrical signal from a signal generator of the IOM system 210 to the electrodes, and by inducing the signal by means of the electrodes into the muscle tissue. The stimulus responses from the stimulated muscle tissue are recorded by means of the conduction electrode(s)/combination electrode(s) of the endotracheal tube 1a, 1b, and/or the separate conduction electrode(s) of the system 20 and are transmitted to the IOM system for displaying as EMGs on a monitor of the IOM system, or for further processing of the stimulus responses.

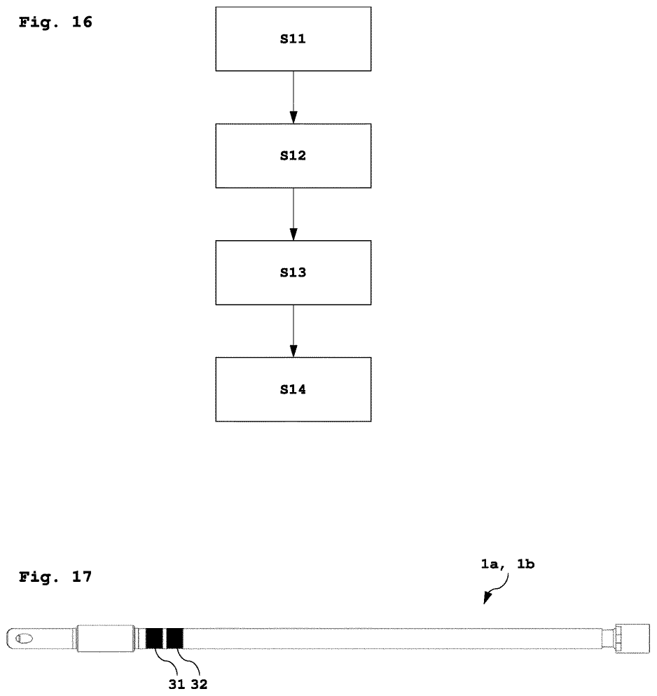

FIG. 16 schematically illustrates the method for classifying tissue types according to the fifth aspect of the present invention. The method for classifying tissue types comprises the steps of inducing S11, measuring S12, calculating S13 and classifying S14. The method can be performed in particular by means of the endotracheal tube 1a, 1b of FIGS. 1a to 12c, or can be performed by the system shown in FIG. 13.

In step S11, at least one alternating electrical signal with at least two different frequencies is induced into tissue by means of at least two inducing electrodes. For this purpose, the at least two inducing electrodes are applied to the tissue. The alternating electrical signal is preferably a sinusoidal signal, square-wave signal, triangular signal or the like in which the at least two frequencies are superimposed, or follow one another in time at defined intervals.

The induced alternating electrical signal causes a current to flow between the inducing electrodes through the tissue to be categorised.

In step S12, a current curve and a voltage curve of the induced alternating electrical signal are measured in the tissue by means of the at least two inducing electrodes or by means of at least two measuring electrodes. The voltage and the current in the tissue resulting from the application of the current of the alternating electrical signal are measured. Preferably, the at least two measuring electrodes are used for this purpose.

In step S13, at least two impedances of the tissue are calculated on the basis of the measured current curve and the measured voltage curve. The at least two impedances are calculated on the basis of the measured current curve and voltage curve at each of the at least two frequencies of the induced alternating electrical signal.

In step S14, a tissue type of the tissue is categorised based on the calculated at least two impedances. For example, a look-up table of impedances at different frequencies and corresponding tissue types can be used to categorise the tissue type based on the calculated impedances of the tissue. In particular, target tissue can be categorised as at least either muscle tissue or other tissue based on the calculated impedances.

FIG. 17 schematically shows an exemplary embodiment of the endotracheal tube 1a, 1b of FIGS. 1 to 12c configured to perform the method for categorizing tissue types shown in FIG. 16. The endotracheal tube 1a, 1b comprises two inducing electrodes 31 and two optional measuring electrodes 32.

The two inducing electrodes 31 are formed by at least two of the stimulation electrodes and/or conduction electrodes and/or combination electrodes of the endotracheal tube 1a, 1b. The two optional measuring electrodes 32 are formed by at least two other of the stimulation electrodes and/or conduction electrodes and/or combination electrodes of the endotracheal tube 1a, 1b.

The alternating electrical signal can be generated by a signal generator or a stimulator to which the inducing electrodes 31 are electrically connectable. The signal generator can be integrated into an IOM system.

The actual measurement of the voltage and/or current can be performed by a measuring unit to which the two optional measuring electrodes 32 can be connected. The measuring unit can be integrated into the IOM system.

A calculation unit, which can be integrated into the IOM system, can calculate the at least two impedances for each of the at least two frequencies of the induced alternating electrical signal from the measured voltage waveform and the measured current waveform.

Subsequently, a categorisation unit, which can be integrated into the IOM system, can perform the categorisation of the tissue type based on the at least two calculated impedances of the tissue.

Although the present invention has been fully described above with reference to preferred embodiments, the present invention not limited thereto, but can be modified in a variety of ways.

List of Reference Signs

1a, 1b Endotracheal tube

2 Tube

3 Fixation element

4 Electrode

4a Stimulation electrode

4b Conduction electrode

4c Combination electrode

5 Electrode array

5a, 5b, 5c, . . . , 5n Sub-arrays

6 Lining

6a Foam structure

6b Spring element

6c Elastic balloon

7 Proximal end

8 Distal end

9 Adhesive electrode

9.1 Electrode section

9.2 Distance indicator

9.3 Electrode connection

10 X-ray contrast strip

11 Depth marker

12 Optics

13 Pressure sensors

14 Separate conduction electrode

15 Counter electrode

20 System

21 Counter electrode

22 Separate conduction electrode

31 Inducing electrode

32 Measuring electrode

100 Patient

110 Trachea

111 Vocal fold

200 Total system

210 IOM system

220 Ventilator

S1 Electrical stimulation

S2 Deriving

S11 Inducing

S12 Measuring

S13 Calculating

S14 Classifying

1. Endotracheal tube (1a) for intra-operative neuromonitoring, comprising:
a tube (2) of substantially circular cross-section configured to be inserted into a trachea of a patient;
a fixation element (3) configured, when the tube (2) is inserted into the trachea of the patient, to fix the tube (2) in the trachea; and
at least two electrodes (4) configured as stimulating electrodes (4a) for electrically stimulating tissue or as deriving electrodes (4b) for deriving stimulus responses from tissue, or configured as combination electrodes (4c) for electrically stimulating tissue and deriving stimulus responses from tissue, and forming at least one electrode array (5) which is arranged at a predefined array distance from a distal end of the tube (2) on the tube outer side and completely surrounds the tube outer side,
wherein the at least two electrodes (4) are arranged in the at least one electrode array (5) such that they are at a predefined electrode spacing from their respective adjacent electrode (4) and are substantially parallel to each other and are circular over 360° [degrees] around the tube (2); wherein the at least two electrodes (4) extend axially or circularly in a predefined range along the tube (2); and wherein the endotracheal tube (1a) comprises:
either at least two stimulation electrodes (4a) and at least two conduction electrodes (4b); or
at least two combination electrodes (4c);
wherein the electrode array (5) is divided into at least two sub-arrays (5a, 5b, 5c); and
wherein the sub-arrays (5a, 5b, 5c) are arranged such that each has a predefined sub-array spacing to its adjacent sub-array (5a, 5b, 5c).

2.-4. (canceled).

5. Endotracheal tube (1a) according to claim 1, further comprising at least one lining (6) associated with at least one of the at least two electrodes (4) and having elasticity in radial direction,
wherein the at least one lining (6) is attached to an underside of the at least one electrode (4) and supports the at least one electrode (4) on the tube exterior such that, under the influence of force in the radial direction, the at least one lining (6) is elastically deformed, and wherein the at least one electrode (4) can be displaced in the radial direction and, when the force is removed, the at least one lining (6) can deform back and the at least one electrode (4) can be displaced back.

6. Endotracheal tube (1a, 1b) according to claim 1, wherein the lining comprises a foam structure (6a) and/or a spring element (6b) and/or an elastic balloon (6c) filled with a gas or gas mixture.

7. Endotracheal tube (1a, 1b) according to claim 1,
wherein at least one of the electrodes (4) or at least one of the paddings (6) with the corresponding at least one electrode (4) is fully integrated into the tube (2), and/or
wherein at least one of the electrodes (4) or at least one of the paddings (6) with the corresponding at least one electrode (4) is formed as an adhesive electrode (9) and is attached to the tube exterior, and/or
wherein at least one of the electrode arrays (5) is formed as an electrode sleeve and is attached tangentially around the tube outer side.

8. Endotracheal tube (1a, 1b) according to claim 7,
wherein the at least one adhesive electrode (9) and/or the at least one electrode cuff comprises a distance indicator (9.2) which is formed to indicate the optimum distance of the at least one adhesive electrode (9) and/or of the at least one electrode cuff to the distal end of the tube (2) or to the fixation element (3).

9. Endotracheal tube (1a, 1b) according to claim 1, further comprising:
an optics (12) configured to visually check the position of the endotracheal tube (1a, 1b) in a trachea and to visually check the vocal cords and their movement.

10. System (20) for intra-operative neuromonitoring, comprising:
an endotracheal tube (1a, 1b) according to claim 1, wherein at least one electrode (4) is configured as a stimulating electrode (4a) for electrically stimulating tissue, or is configured as a combination electrode (4c) for electrically stimulating tissue and deriving stimulus responses from the tissue; and
at least one separate conduction electrode (14) configured to be attached on the patient in proximity to the stimulated tissue and to derive stimulation responses from the stimulated tissue.

11. System (20) according to claim 10, further comprising:
at least one counter electrode (15) configured to be externally attached to a patient, and configured to stimulate, in combination with the at least one stimulation electrode (4a) or the at least one combination electrode (4c), the tissue in a monopolar manner.

12. System (10) according to claim 10,
wherein the at least one counter electrode (15) is formed as a needle electrode or as a surface electrode, and is formed to stimulate tissue subcutaneously or transcutaneously in combination with the at least one stimulation electrode (4a) or the at least one combination electrode (4c).

13. Method for deriving stimulus responses in intra-operative neuromonitoring comprising an endotracheal tube, the method comprising the steps of:
electrically stimulating (S1) tissue by means of at least one stimulation electrode (4a) or at least one combination electrode (4c) of the endotracheal tube (1a, 1b); and
deriving (S2) a stimulus response of the stimulated tissue by means of at least one conduction electrode (4b) or at least one combination electrode (4c) of the endotracheal tube (1a, 1b), or by means of at least one separate conduction electrode (14) of the system (20).

14. Method according to claim 13,
wherein the step of electrically stimulating (S1) of tissue is additionally performed by means of at least one counter electrode (15) of the system (20).

15. Method according to claim 13,
wherein the step of electrically stimulating (S1) is performed in a monopolar fashion by means of the at least one stimulation electrode (4a) or the at least one combination electrode (4c) of the endotracheal tube (1a, 1b) and the at least one counter electrode (15) of the system (10), is performed in a bipolar fashion by means of at least two stimulation electrodes (4a) and/or combination electrodes (4c) of the endotracheal tube (1a, 1b), or is performed in a multipolar fashion by means of multiple stimulation electrodes (4a) and/or combination electrodes (4c) of the endotracheal tube (1a, 1b).

16. Method of classifying tissue types, comprising the steps of:
inducing (S11) at least one alternating electrical signal with at least two different predefined frequencies into tissue by means of at least two inducing electrodes (31);
measuring (S12) a current waveform and a voltage waveform of the induced alternating electrical signal in the tissue by means of the at least two inducing electrodes (31) or by means of at least two measuring electrodes (32);
calculating (S13) at least two impedances of the tissue by means of the measured current waveform and the measured voltage waveform; and
classifying (S14) a tissue type of the tissue based on the calculated at least two impedances, wherein the at least two inducing electrodes (31) are formed by stimulation electrodes (4a), inducing electrodes (4b), or combination electrodes (4c) on a tube outer side of a tube (2) of an endotracheal tube (1a, 1b).

17. (canceled).

18. Endotracheal tube for classifying tissue types configured to be able to perform the method according to claim 16, wherein the at least two inducing electrodes (13) and optionally the at least two measuring electrodes (14) are arranged on a tube outer side of a tube of the endotracheal tube.

19. Endotracheal tube (1a, 1b) according to claim 1, wherein at least two of the electrodes (4) are the at least two inducing electrodes (31), and optionally wherein at least two further ones of the electrodes (4) are the at least two measuring electrodes (32).

20. System (20) according to claim 10, wherein at least two of the electrodes (4) are either the at least two inducing electrodes (31) or are the at least two measuring electrodes (32), and wherein at least two separate deduction electrodes (14) or counter electrodes (15) are, respectively, either the at least two measuring electrodes (32) or the at least two inducing electrodes (31).
